(19) United States
(12) Patent Application Publication
(10) Pub. No.: US 2018/0125500 A1
(43) Pub. Date: May 10, 2018

(54) Intrasacular Aneurysm Occlusion Device with Mesh-Filled Loops

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 15/861,482

(22) Filed: Jan. 3, 2018

Related U.S. Application Data
(63) Continuation-in-part of application No. 14/526,600, filed on Oct. 29, 2014.
Application No. 14/526,600 is a continuation-in-part of application No. 12/989,048, filed on Oct. 21, 2010, now Pat. No. 8,974,487.
Application No. 12/989,048 is a continuation-in-part of application No. 15/080,915, filed on Mar. 25, 2016.
Application No. 15/080,915 is a continuation-in-part of application No. 14/526,600, filed on Oct. 29, 2014.
Application No. 14/526,600 is a continuation-in-part of application No. 12/989,048, filed on Oct. 21, 2010, now Pat. No. 8,974,487.
(60) Provisional application No. 61/126,047, filed on May 1, 2008.
(60) Provisional application No. 61/126,027, filed on May 1, 2008.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.
(60) Provisional application No. 62/444,860, filed on Jan. 11, 2017.
(60) Provisional application No. 62/472,519, filed on Mar. 16, 2017.
(60) Provisional application No. 62/589,754, filed on Nov. 22, 2017.

(51) Int. Cl. A61B 17/12

(52) U.S. Cl. CPC A61B 17/12113; A61B 17/12031; A61B 2017/00862; A61B 17/12168; A61B 17/12145; A61B 17/1214

(57) ABSTRACT

This invention is an intrasacular aneurysm occlusion device comprising: two longitudinal wires which are inserted into an aneurysm sac; and mesh which spans between the wires. In an example, the wires can be sinusoidal. In an example, the wires can intersect multiple times to form loops which are spanned by mesh. In an example, the wires can converge and diverge multiple times to form arcuate areas between them which are spanned by mesh. In an example, the device can further comprise a third wire between the first and second wires.

### CROSS-REFERENCE TO RELATED APPLICATIONS

This patent application:

(1) is a continuation in part of U.S. patent application Ser. No. 14/526,600 entitled “Devices and Methods for Occluding a Cerebral Aneurysm” by Robert A. Connor which was filed on Oct. 29, 2014—which in turn was a continuation in part of U.S. patent application Ser. No. 12/989,048 entitled “Aneurysm Occlusion Device” by Robert A. Connor and Muhammad Tariq Janjua which has a 371 date of Oct. 21, 2010, a filing date of Apr. 24, 2009, and a priority date of May 1, 2008 which is the U.S. national phase filing of PCT/US 2009/002537 entitled “Aneurysm Occlusion Device” by Robert A. Connor and Muhammad Tariq Janjua filed on Apr. 24, 2009 which claimed the priority benefit of U.S. Provisional Patent Application 61/126,047 entitled “Flow of Soft Members into a Net to Embolize an Aneurysm” by Robert A. Connor which received a filing date of May 1, 2008 and claimed the priority benefit of U.S. Provisional Patent Application 61/126,027 entitled “Net Filled with Soft Members to Embolize an Aneurysm” by Robert A. Connor which received a filing date of May 1, 2008; and also claimed the priority benefit of U.S. Provisional Patent Application 61/897,245 entitled “Devices and Methods for Occluding a Cerebral Aneurysm” by Robert A. Connor filed on Oct. 30, 2013;

(2) is a continuation in part of U.S. patent application Ser. No. 15/080,915 entitled “Coils with a Series of Proximally-and-Distally-Connected Loops for Occluding a Cerebral Aneurysm” by Robert A. Connor which was filed on Mar. 25, 2016—which in turn was a continuation in part of U.S. patent application Ser. No. 14/526,600 entitled “Devices and Methods for Occluding a Cerebral Aneurysm” by Robert A. Connor which was filed on Oct. 29, 2014 and was a continuation in part of U.S. patent application Ser. No. 12/989,048 entitled “Aneurysm Occlusion Device” by Robert A. Connor and Muhammad Tariq Janjua which has a 371 date of Oct. 21, 2010, a filing date of Apr. 24, 2009, and a priority date of May 1, 2008 which is the U.S. national phase filing of PCT/US 2009/002537 entitled “Aneurysm Occlusion Device” by Robert A. Connor and Muhammad Tariq Janjua filed on Apr. 24, 2009 which claimed the priority benefit of U.S. Provisional Patent Application No. 61/126,047 entitled “Flow of Soft Members into a Net to Embolize an Aneurysm” by Robert A. Connor which received a filing date of May 1, 2008 and claimed the priority benefit of U.S. Provisional Patent Application No. 61/126,027 entitled “Net Filled with Soft Members to Embolize an Aneurysm” by Robert A. Connor which received a filing date of May 1, 2008; and also claimed the priority benefit of U.S. Provisional Patent Application 61/897,245 entitled “Devices and Methods for Occluding a Cerebral Aneurysm” by Robert A. Connor filed on Oct. 30, 2013;

(3) claims the priority benefit of U.S. Provisional Patent Application 62/444,860 entitled “Aneurysm Occlusion Device with Undulating Longitudinal Segments” by Robert A. Connor filed on Jan. 11, 2017;

(4) claims the priority benefit of U.S. Provisional Patent Application 62/472,519 entitled “Devices for Occluding a Cerebral Aneurysm” by Robert A. Connor filed on Mar. 16, 2017; and

(5) claims the priority benefit of U.S. Provisional Patent Application 62/589,754 entitled “Intrasacular Aneurysm Occlusion Device with a Resilient Wider-Than-Neck Portion and a Flexible Sac-Filling Portion” by Robert A. Connor filed on Nov. 22, 2017.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices and methods for occluding a blood vessel aneurysm.

### Introduction to Cerebral Aneurysms

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain.

Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function.

### Review of the Most Relevant Art

U.S. Patent Application Publications 20120239074 (Aboytes et al., Sep. 20, 2012, “Devices and Methods for the Treatment of Vascular Defects”), 20150209050 (Aboytes et al., Jul. 30, 2015, “Devices and Methods for the Treatment of Vascular Defects”), and 20160262766 (Aboytes et al., Sep. 15, 2016, “Devices and Methods for the Treatment of Vascular Defects”) disclose an intrasacular aneurysm occlusion device comprising an expandable implant with a first configuration in which the first portion and the second portion are substantially linearly aligned and a second configuration in which the second portion at least partially overlaps the first portion.

U.S. Patent Application Publications 20150297240 (Divino et al., Oct. 22, 2015, “Embolic Medical Devices”) and 20170281194 (Divino et al., Oct. 5, 2017, “Embolic Medical Devices”) disclose an intrasacular aneurysm occlusion device with a collapsed configuration in which its first and second side edges are curled toward each other around a longitudinal axis and an expanded configuration forming a series of loops wherein the first and second side edges uncurl. U.S. Patent Application Publication 20170079662 (Rhee et al., Mar. 23, 2017, “Occlusive Devices”) discloses an aneurysm occlusion device comprising frame and mesh components, wherein the frame and mesh components have different porosity levels.

U.S. Patent Application Publication 20170189035 (Porter, Jul. 6, 2017, “Embolic Devices and Methods of Manufacturing Same”) discloses an intrasacular aneurysm occlusion device comprising a flat embolic braid having a first side comprising a first side surface and a second side comprising a second side surface facing in an opposite direction than the first side surface, the braid having an elongated constrained configuration for being deployed through a delivery catheter, and a three-dimensional unconstrained configuration, wherein in the three-dimensional unconstrained configuration, the braid assumes a plurality of successive loops in which the braid is at least partially twisted between successive loops of the plurality, so that the first side surface faces externally of each loop, and the second side surface faces an interior of each loop, respectively, regardless of a change in direction and/or orientation of the braid.

U.S. Patent Application Publications 20160249935 (Hewitt et al., Sep. 1, 2016, “Devices for Therapeutic Vascular Procedures”) and 20160367260 (Hewitt et al., Dec. 22, 2016, “Devices for Therapeutic Vascular Procedures”) disclose an intrasacular aneurysm occlusion device comprising a distal self-expanding resilient permeable shell, a proximal self-expanding resilient permeable shell, and an elongate support member between the distal and proximal permeable shells. U.S. Patent Application Publication 20170095254 (Hewitt et al., May 6, 2017, “Filamentary Devices for Treatment of Vascular Defects”) discloses an aneurysm occlusion device comprising a self-expanding permeable shell having a radially constrained elongated state configured for delivery within a catheter lumen, an expanded state with a globular and longitudinally shortened configuration relative to the radially constrained state, and a plurality of elongate filaments that are woven together, which define a cavity of the permeable shell. U.S. Patent Application Publication 20170128077 (Hewitt et al., May 11, 2017, “Devices for Therapeutic Vascular Procedures”) discloses an aneurysm occlusion device comprising a self-expanding resilient permeable shell and a metallic coil secured to the distal end of a shell. U.S. Patent Application Publication 20170128077 (Hewitt et al., May 11, 2017, “Devices for Therapeutic Vascular Procedures”) discloses an aneurysm occlusion device comprising an expandable cylindrical structure made of wires and a self-expanding permeable shell located at the distal end of the cylindrical structure.

U.S. Patent Application Publication 20170086851 (Wallace et al., Mar. 30, 2017, “Vaso-Occlusive Devices and Methods of Use”) discloses expandable vaso-occlusive implants that include one or more soft and expandable braided members coupled to a pushable member such as a coil that maybe inserted and retrieved from within an aneurism using a delivery catheter. U.S. Patent Application Publication 20170156733 (Becking et al., Jun. 8, 2017, “Multiple Layer Filamentary Devices for Treatment of Vascular Defects”) discloses braid balls for aneurysm occlusion and/or parent vessel occlusion/sacrifice.

### SUMMARY OF THE INVENTION

This invention is an intrasacular aneurysm occlusion device comprising two longitudinal wires with mesh between them. The device is inserted into an aneurysm sac. As the device accumulates within the aneurysm sac, it forms a mass which covers the aneurysm neck from the inside and reduces blood flow into the aneurysm sac. This invention can comprise a first longitudinal wire which is inserted into an aneurysm sac, a second longitudinal wire which is inserted into the aneurysm sac, and mesh which spans between the wires. In an example, the wires can be sinusoidal. In an example, the wires can intersect multiple times to form loops between them, wherein these loops are spanned by mesh. In an example, the wires can converge and diverge multiple times to form areas between them, wherein these areas are spanned by the mesh. In an example, the device can further comprise a third wire between the first and second wires.

### INTRODUCTION TO THE FIGURES

FIGS. 1 through 3 show an intrasacular aneurysm occlusion device with a series of connected wire loops.

FIGS. 4 through 6 show an intrasacular aneurysm occlusion device with a series of connected mesh-filled wire loops.

FIG. 7 shows an intrasacular aneurysm occlusion device with mesh-filled loops formed by intersecting or overlapping first and second wires.

FIG. 8 shows an intrasacular aneurysm occlusion device with mesh-filled loops formed by converging and diverging first and second wires.

FIG. 9 shows an intrasacular aneurysm occlusion device with mesh-filled loops which are bilaterally connected by elastic material.

FIG. 10 shows an intrasacular aneurysm occlusion device with mesh-filled loops which are unilaterally connected by elastic material.

FIG. 11 shows an intrasacular aneurysm occlusion device with mesh-filled loops which are connected on alternating sides by elastic material.

FIG. 12 shows an intrasacular aneurysm occlusion device with mesh-filled loops which are bilaterally connected by springs.

FIG. 13 shows a first intrasacular aneurysm occlusion device with mesh-filled loops which are bilaterally connected by pull cords.

FIG. 14 shows a second intrasacular aneurysm occlusion device with mesh-filled loops which are bilaterally connected by pull cords.

FIG. 15 shows an intrasacular aneurysm occlusion device with asymmetric undulating mesh-filled loops.

FIG. 16 shows an intrasacular aneurysm occlusion device with alternating asymmetric undulating mesh-filled loops.

FIG. 17 shows an intrasacular aneurysm occlusion device with a series of mesh-filled loops and a central sinusoidal wire.

FIG. 18 shows an intrasacular aneurysm occlusion device with a series of mesh-filled loops and a central compound sinusoidal wire.

FIG. 19 shows an intrasacular aneurysm occlusion device with a series of concentric mesh-filled loops.

FIG. 20 shows an intrasacular aneurysm occlusion device with a series of mesh-filled loops with lateral springs.

FIG. 21 shows an intrasacular aneurysm occlusion device with a series of mesh-filled loops with a central straight longitudinal wire.

FIG. 22 shows an intrasacular aneurysm occlusion device with a series of wide mesh-filled loops connected by narrow mesh-filled segments.

FIG. 23 shows an intrasacular aneurysm occlusion device with a series of alternating single-phase-sinusoidal mesh-filled loops.

FIG. 24 shows an intrasacular aneurysm occlusion device with two parallel undulating wires connected by a mesh.

FIG. 25 shows an intrasacular aneurysm occlusion device with two straight wires connected by a mesh and a sinusoidal central wire.

FIG. 26 shows an intrasacular aneurysm occlusion device with two overlapping out-of-phase-sinusoidal wires and a mesh between them.

FIG. 27 shows a first intrasacular aneurysm occlusion device with two non-overlapping out-of-phase-sinusoidal wires and a mesh between them.

FIG. 28 shows a second intrasacular aneurysm occlusion device with two non-overlapping out-of-phase-sinusoidal wires and a mesh between them.

FIG. 29 shows an intrasacular aneurysm occlusion device with two sinusoidal wires with different amplitudes and a mesh between them.

FIG. 30 shows an intrasacular aneurysm occlusion device with laterally-alternating sinusoidal mesh-filled segments.

FIG. 31 shows an intrasacular aneurysm occlusion device with a series of laterally-alternating crescent-shaped mesh-filled segments.

FIG. 32 shows an intrasacular aneurysm occlusion device with a series of concentric elliptical mesh-filled loops.

FIG. 33 shows an intrasacular aneurysm occlusion device with a series of laterally-alternating bowl-shaped mesh-filled segments.

FIG. 34 shows an intrasacular aneurysm occlusion device with a series of collateral bowl-shaped mesh-filled segments.

FIG. 35 shows an intrasacular aneurysm occlusion device with two sinusoidal wires and laterally-alternating asymmetric mesh-filled loops.

FIG. 36 shows an intrasacular aneurysm occlusion device with two sinusoidal wires and collateral asymmetric mesh-filled loops.

FIG. 37 shows an intrasacular aneurysm occlusion device with two sinusoidal wires and bilateral elastic connectors.

FIG. 38 shows an intrasacular aneurysm occlusion device with two sinusoidal wires and unilateral elastic connectors.

FIG. 39 shows an intrasacular aneurysm occlusion device with two sinusoidal wires and laterally-alternating elastic connectors.

FIG. 40 shows an intrasacular aneurysm occlusion device with three parallel sinusoidal wires connected by mesh.

FIG. 41 shows an intrasacular aneurysm occlusion device with three out-of-phase-sinusoidal wires connected by mesh.

FIG. 42 shows a first intrasacular aneurysm occlusion device with two straight wires, a central sinusoidal wire, and different types of mesh between them.

FIG. 43 shows a second intrasacular aneurysm occlusion device with two straight wires, a central sinusoidal wire, and different types of mesh between them.

FIG. 44 shows an intrasacular aneurysm occlusion device with inter-twined undulating bands.

FIG. 45 shows a first intrasacular aneurysm occlusion device with four sinusoidal wires connected by mesh.

FIG. 46 shows a second intrasacular aneurysm occlusion device with four sinusoidal wires connected by mesh.

FIGS. 47 and 48 show stand-alone and sac-deployed views of an intrasacular aneurysm occlusion device with inter-twined sinusoidal bands.

FIGS. 49 and 50 show stand-alone and sac-deployed views of an intrasacular aneurysm occlusion device with three coaxial sinusoidal wires connected by mesh.

FIGS. 51 and 52 show stand-alone and sac-deployed views of an intrasacular aneurysm occlusion device with two sinusoidal wires with different frequencies connected by mesh.

FIGS. 53 and 54 show stand-alone and sac-deployed views of an intrasacular aneurysm occlusion device with two catenary-or-semicircle-sequence wires connected by mesh.

FIGS. 55 and 56 show stand-alone and sac-deployed views of an intrasacular aneurysm occlusion device with cardioid or kidney shaped mesh-filled loops.

FIGS. 57 and 58 show stand-alone and sac-deployed views of an intrasacular aneurysm occlusion device with two out-of-phase-sinusoidal wires connected by mesh.

### DETAILED DESCRIPTION OF THE FIGURES

In an example, a device for occluding an aneurysm can comprise: a first arcuate wire which is inserted into an aneurysm sac; a second arcuate wire which is inserted into the aneurysm sac; and a net or mesh which spans at least one area between the first arcuate wire and the second arcuate wire. In an example, a device for occluding an aneurysm can comprise: a first undulating and/or sinusoidal wire which is inserted into an aneurysm sac; a second undulating and/or sinusoidal wire which is inserted into the aneurysm sac; and a net or mesh which spans at least one area between the first undulating and/or sinusoidal wire and the second undulating and/or sinusoidal wire. In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least twice along their longitudinal axes forming at least one area with a wire perimeter between the first arcuate wire and the second arcuate wire; and a net or mesh which spans the area. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least twice along their longitudinal axes forming at least one area with a wire perimeter between the first undulating and/or sinusoidal wire and the second undulating and/or sinusoidal wire; and a net or mesh which spans the area.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least twice along their longitudinal axes forming at least one area with a wire perimeter between the first arcuate wire and the second arcuate wire; and a net or mesh which spans the area. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least twice along their longitudinal axes forming at least one area with a wire perimeter between the first undulating and/or sinusoidal wire and the second undulating and/or sinusoidal wire; and a net or mesh which spans the area.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least twice along their longitudinal axes forming at least one area with a wire perimeter between the first arcuate wire and the second arcuate wire, wherein a first portion of the wire perimeter is a portion of the first arcuate wire, and wherein a second portion of the wire perimeter is a portion of the second arcuate wire; and a net or mesh which spans the area. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least twice along their longitudinal axes forming at least one area with a wire perimeter between the first undulating and/or sinusoidal wire and the second undulating and/or sinusoidal wire, wherein a first portion of the wire perimeter is a portion of the first undulating and/or sinusoidal wire, and wherein a second portion of the wire perimeter is a portion of the second undulating and/or sinusoidal wire; and a net or mesh which spans the area.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least twice along their longitudinal axes forming at least one area with a wire perimeter between the first arcuate wire and the second arcuate wire, wherein a first portion of the wire perimeter is a portion of the first arcuate wire, and wherein a second portion of the wire perimeter is a portion of the second arcuate wire; and a net or mesh which spans the area. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least twice along their longitudinal axes forming at least one area with a wire perimeter between the first undulating and/or sinusoidal wire and the second undulating and/or sinusoidal wire, wherein a first portion of the wire perimeter is a portion of the first undulating and/or sinusoidal wire, and wherein a second portion of the wire perimeter is a portion of the second undulating and/or sinusoidal wire; and a net or mesh which spans the area.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least twice along their longitudinal axes forming at least one wire loop between the first arcuate wire and the second arcuate wire; and a net or mesh which spans the loop. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least twice along their longitudinal axes forming at least one wire loop between the first undulating and/or sinusoidal wire and the second undulating and/or sinusoidal wire; and a net or mesh which spans the loop.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least twice along their longitudinal axes forming at least one wire loop between the first arcuate wire and the second arcuate wire; and a net or mesh which spans the loop. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least twice along their longitudinal axes forming at least one wire loop between the first undulating and/or sinusoidal wire and the second undulating and/or sinusoidal wire; and a net or mesh which spans the loop.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least twice along their longitudinal axes forming at least one wire loop between the first arcuate wire and the second arcuate wire, wherein a first portion of the wire loop is a portion of the first arcuate wire, and wherein a second portion of the wire loop is a portion of the second arcuate wire; and a net or mesh which spans the loop. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least twice along their longitudinal axes forming at least one wire loop between the first undulating and/or sinusoidal wire and the second undulating and/or sinusoidal wire, wherein a first portion of the wire loop is a portion of the first undulating and/or sinusoidal wire, and wherein a second portion of the wire loop is a portion of the second undulating and/or sinusoidal wire; and a net or mesh which spans the loop.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least twice along their longitudinal axes forming at least one wire loop between the first arcuate wire and the second arcuate wire, wherein a first portion of the wire loop is a portion of the first arcuate wire, and wherein a second portion of the wire loop is a portion of the second arcuate wire; and a net or mesh which spans the loop. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least twice along their longitudinal axes forming at least one wire loop between the first undulating and/or sinusoidal wire and the second undulating and/or sinusoidal wire, wherein a first portion of the wire loop is a portion of the first undulating and/or sinusoidal wire, and wherein a second portion of the wire loop is a portion of the second undulating and/or sinusoidal wire; and a net or mesh which spans the loop.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least three times along their longitudinal axes forming at least two areas with wire perimeters between the first arcuate wire and the second arcuate wire; and nets or meshes which span the areas. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least three times along their longitudinal axes forming at least two areas with wire perimeters between the first undulating and/or sinusoidal wire and the second undulating and/or sinusoidal wire; and nets or meshes which span the areas. In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least three times along their longitudinal axes forming at least two areas with wire perimeters between the first arcuate wire and the second arcuate wire; and nets or meshes which span the areas. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least three times along their longitudinal axes forming at least two areas with wire perimeters between the first undulating and/or sinusoidal wire and the second undulating and/or sinusoidal wire; and nets or meshes which span the areas.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least three times along their longitudinal axes forming at least two areas with wire perimeters between the first arcuate wire and the second arcuate wire, wherein a first portion of a wire perimeter is a portion of the first arcuate wire, and wherein a second portion of a wire perimeter is a portion of the second arcuate wire; and nets or meshes which span the areas. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least three times along their longitudinal axes forming at least two areas with wire perimeters between the first undulating and/or sinusoidal wire and the second undulating and/or sinusoidal wire, wherein a first portion of a wire perimeter is a portion of the first undulating and/or sinusoidal wire, and wherein a second portion of a wire perimeter is a portion of the second undulating and/or sinusoidal wire; and nets or meshes which span the areas.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least three times along their longitudinal axes forming at least two areas with wire perimeters between the first arcuate wire and the second arcuate wire, wherein a first portion of a wire perimeter is a portion of the first arcuate wire, and wherein a second portion of a wire perimeter is a portion of the second arcuate wire; and nets or meshes which span the areas. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least three times along their longitudinal axes forming at least two areas with wire perimeters between the first undulating and/or sinusoidal wire and the second undulating and/or sinusoidal wire, wherein a first portion of a wire perimeter is a portion of the first undulating and/or sinusoidal wire, and wherein a second portion of a wire perimeter is a portion of the second undulating and/or sinusoidal wire; and nets or meshes which span the areas.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least three times along their longitudinal axes forming at least two wire loops between the first arcuate wire and the second arcuate wire; and nets or meshes which span the loops. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least three times along their longitudinal axes forming at least two wire loops between the first undulating and/or sinusoidal wire and the second undulating and/or sinusoidal wire; and nets or meshes which span the loops. In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least three times along their longitudinal axes forming at least two wire loops between the first arcuate wire and the second arcuate wire; and nets or meshes which span the loops. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least three times along their longitudinal axes forming at least two wire loops between the first undulating and/or sinusoidal wire and the second undulating and/or sinusoidal wire; and nets or meshes which span the loops.

In an example, a device for occluding an aneurysm can comprise: a first arcuate wire which is inserted into an aneurysm sac; a second arcuate wire which is inserted into the aneurysm sac, wherein the first arcuate wire and the second arcuate wire intersect and/or overlap at least three times along their longitudinal axes forming at least two wire loops between the first arcuate wire and the second arcuate wire, wherein a first portion of the wire loop is a portion of the first arcuate wire, and wherein a second portion of the wire loop is a portion of the second arcuate wire; and nets or meshes which span the loops. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire intersect and/or overlap at least three times along their longitudinal axes forming at least two wire loops between the first undulating and/or sinusoidal wire and the second undulating and/or sinusoidal wire, wherein a first portion of the wire loop is a portion of the first undulating and/or sinusoidal wire, and wherein a second portion of the wire loop is a portion of the second undulating and/or sinusoidal wire; and nets or meshes which span the loops.

In an example, a device for occluding an aneurysm can comprise: a first arcuate wire which is inserted into an aneurysm sac; a second arcuate wire which is inserted into the aneurysm sac, wherein the first arcuate wire and the second arcuate wire converge and diverge at least three times along their longitudinal axes forming at least two wire loops between the first arcuate wire and the second arcuate wire, wherein a first portion of the wire loop is a portion of the first arcuate wire, and wherein a second portion of the wire loop is a portion of the second arcuate wire; and nets or meshes which span the loops. In an example, a device for occluding an aneurysm can comprise: a first undulating and/or sinusoidal wire which is inserted into an aneurysm sac; a second undulating and/or sinusoidal wire which is inserted into the aneurysm sac, wherein the first undulating and/or sinusoidal wire and the second undulating and/or sinusoidal wire converge and diverge at least three times along their longitudinal axes forming at least two wire loops between the first undulating and/or sinusoidal wire and the second undulating and/or sinusoidal wire, wherein a first portion of the wire loop is a portion of the first undulating and/or sinusoidal wire, and wherein a second portion of the wire loop is a portion of the second undulating and/or sinusoidal wire; and nets or meshes which span the loops.

In an example, a device for occluding an aneurysm can comprise: a first arcuate wire which is inserted into an aneurysm sac; a second arcuate wire which is inserted into the aneurysm sac; a third arcuate wire which is inserted into the aneurysm sac, wherein the second arcuate wire is between the first arcuate wire and the third arcuate wire; a first net or mesh with a first elasticity level which spans at least one area between the first arcuate wire and the second arcuate wire; a second net or mesh with a second elasticity level which spans at least one area between the second arcuate wire and the third arcuate wire, wherein the second elasticity level is different than the first elasticity level. In an example, a device for occluding an aneurysm can comprise: a first undulating and/or sinusoidal wire which is inserted into an aneurysm sac; a second undulating and/or sinusoidal wire which is inserted into the aneurysm sac; a third undulating and/or sinusoidal wire which is inserted into the aneurysm sac, wherein the second undulating and/or sinusoidal wire is between the first undulating and/or sinusoidal wire and the third undulating and/or sinusoidal wire; a first net or mesh with a first elasticity level which spans at least one area between the first undulating and/or sinusoidal wire and the second undulating and/or sinusoidal wire; a second net or mesh with a second elasticity level which spans at least one area between the second undulating and/or sinusoidal wire and the third undulating and/or sinusoidal wire, wherein the second elasticity level is different than the first elasticity level.

In an example, a device for occluding an aneurysm can comprise: a first arcuate wire which is inserted into an aneurysm sac; a second arcuate wire which is inserted into the aneurysm sac; a third arcuate wire which is inserted into the aneurysm sac, wherein the second arcuate wire is between the first arcuate wire and the third arcuate wire; a first net or mesh with a first density which spans at least one area between the first arcuate wire and the second arcuate wire; a second net or mesh with a second density which spans at least one area between the second arcuate wire and the third arcuate wire, wherein the second density is different than the first density. In an example, a device for occluding an aneurysm can comprise: a first undulating and/or sinusoidal wire which is inserted into an aneurysm sac; a second undulating and/or sinusoidal wire which is inserted into the aneurysm sac; a third undulating and/or sinusoidal wire which is inserted into the aneurysm sac, wherein the second undulating and/or sinusoidal wire is between the first undulating and/or sinusoidal wire and the third undulating and/or sinusoidal wire; a first net or mesh with a first density which spans at least one area between the first undulating and/or sinusoidal wire and the second undulating and/or sinusoidal wire; a second net or mesh with a second density which spans at least one area between the second undulating and/or sinusoidal wire and the third undulating and/or sinusoidal wire, wherein the second density is different than the first density.

In an example, a device for occluding an aneurysm can comprise: a first arcuate wire which is inserted into an aneurysm sac; a second arcuate wire which is inserted into the aneurysm sac; a third arcuate wire which is inserted into the aneurysm sac, wherein the second arcuate wire is between the first arcuate wire and the third arcuate wire; a first net or mesh made from a first material which spans at least one area between the first arcuate wire and the second arcuate wire; a second net or mesh made from a second material spans at least one area between the second arcuate wire and the third arcuate wire, wherein the second material is different than the first material. In an example, a device for occluding an aneurysm can comprise: a first undulating and/or sinusoidal wire which is inserted into an aneurysm sac; a second undulating and/or sinusoidal wire which is inserted into the aneurysm sac; a third undulating and/or sinusoidal wire which is inserted into the aneurysm sac, wherein the second undulating and/or sinusoidal wire is between the first undulating and/or sinusoidal wire and the third undulating and/or sinusoidal wire; a first net or mesh made from a first material which spans at least one area between the first undulating and/or sinusoidal wire and the second undulating and/or sinusoidal wire; a second net or mesh made from a second material spans at least one area between the second undulating and/or sinusoidal wire and the third undulating and/or sinusoidal wire, wherein the second material is different than the first material.

In an example, a device for occluding an aneurysm can comprise a flexible net or mesh which is inserted into an aneurysm. In an example, a device for occluding an aneurysm can comprise: a multiple-width longitudinal mesh which is configured to be inserted into an aneurysm sac; wherein the multiple-width longitudinal mesh has a distal-to-proximal longitudinal axis prior to insertion into the aneurysm sac, wherein the multiple-width longitudinal mesh has a length dimension along (or parallel to) the distal-to-proximal longitudinal axis, wherein the multiple-width longitudinal mesh has a width dimension perpendicular (or orthogonal) to the length dimension, and wherein the multiple-width longitudinal mesh has a thickness dimension perpendicular (or orthogonal) to the length dimension and the width dimension; wherein the multiple-width longitudinal mesh further comprises a plurality of narrow longitudinal segments with a first average length, a first average width, and a first average thickness; wherein the multiple-width longitudinal mesh further comprises a plurality of wide longitudinal segments with a second average length, a second average width, and a second average thickness; and wherein the second average width is at least 50% more than the first average width, wherein the second average width is at least twice the second average thickness, wherein the second average length is at least equal to the first average length, and wherein these three dimensional comparisons are made after the multiple-width longitudinal mesh has been inserted into the aneurysm sac.

In an example, this device can create an occluding arcuate mass of interconnected contiguous mesh loops within an aneurysm. In an example, narrow longitudinal segments and wide longitudinal segments can be contiguous with each other. In an example, the second average width can be at least twice the first average width after the multiple-width longitudinal mesh has been inserted into the aneurysm sac. In an example, the second average width can be at least four times the second average thickness after the multiple-width longitudinal mesh has been inserted into the aneurysm sac. In an example, the maximum width among wide longitudinal segments can be at least twice the maximum width among narrow longitudinal segments after the multiple-width longitudinal mesh has been inserted into the aneurysm sac. In an example, the maximum width among wide longitudinal segments can be at least twice the maximum thickness among wide longitudinal segments after the multiple-width longitudinal mesh has been inserted into the aneurysm sac.

In an example, a device for occluding an aneurysm can comprise: a multiple-width longitudinal mesh which is configured to be inserted into an aneurysm sac; wherein the multiple-width longitudinal mesh has a first configuration prior to insertion into the aneurysm sac and a second configuration after insertion into aneurysm sac; wherein the multiple-width longitudinal mesh has a distal-to-proximal longitudinal axis in the first configuration; wherein the multiple-width longitudinal mesh has a length dimension along (or parallel to) the distal-to-proximal longitudinal axis, wherein the multiple-width longitudinal mesh has a width dimension perpendicular (or orthogonal) to the length dimension, and wherein the multiple-width longitudinal mesh has a thickness dimension perpendicular (or orthogonal) to the length dimension and the width dimension; wherein the multiple-width longitudinal mesh further comprises a plurality of narrow longitudinal segments with a first average length, a first average width, and a first average thickness; wherein the multiple-width longitudinal mesh further comprises a plurality of wide longitudinal segments with a second average length, a second average width, and a second average thickness; and wherein the second average width is at least 50% more than the first average width in the second configuration; wherein the second average width is at least twice the second average thickness in the second configuration; and wherein the second average length is at least equal to the first average length in the second configuration.

In an example, this device can create an occluding arcuate mass of interconnected contiguous mesh loops within an aneurysm. In an example, narrow longitudinal segments and wide longitudinal segments can be contiguous with each other. In an example, the second average width can be at least twice the first average width in the second configuration. In an example, the second average width can be at least four times the second average thickness in the second configuration. In an example, the maximum width among wide longitudinal segments can be at least twice the maximum width among narrow longitudinal segments in the second configuration. In an example, the maximum width among wide longitudinal segments can be at least twice the maximum thickness among wide longitudinal segments in the second configuration.

In an example, a (first and/or second) longitudinal segment can further comprise: a first (e.g. right side) longitudinal wire (or coil, strand, or fiber); a second (e.g. left-side) longitudinal wire (or coil, strand, or fiber); and an inner mesh section (or net or low-porosity barrier) spanning between the first and second longitudinal wires. In an example, a continuous first longitudinal wire and/or second longitudinal wire can be part of two or more longitudinal segments. In an example, each longitudinal segment can have a separate first longitudinal wire and/or second longitudinal wire.

In an example, a device for occluding an aneurysm can comprise: a plurality of connected longitudinal segments which is inserted into an aneurysm sac; wherein a longitudinal segment further comprises a first longitudinal wire comprising one (e.g. the right) side of the longitudinal segment, a second longitudinal wire comprising the opposite (e.g. the left) side of the longitudinal segment, and an inner mesh section spanning between the first longitudinal wire and the second longitudinal wire; wherein a longitudinal segment has a first configuration with a first width prior to insertion into the aneurysm sac and a second configuration with a second width after insertion into aneurysm sac; and wherein the second width is greater than the first width.

In an example, a device for occluding an aneurysm can comprise: a plurality of connected longitudinal segments which is inserted into an aneurysm sac; wherein each longitudinal segment further comprises a first longitudinal wire comprising one (e.g. the right) side of the longitudinal segment, a second longitudinal wire comprising the opposite (e.g. the left) side of the longitudinal segment, and an inner mesh section spanning between the first longitudinal wire and the second longitudinal wire; wherein each longitudinal segment has a first configuration with a first width prior to insertion into the aneurysm sac and a second configuration with a second width after insertion into aneurysm sac; and wherein the second width is greater than the first width.

In an example, this device can further comprise a first longitudinal section of a flexible longitudinal embolic member and a second longitudinal section of a flexible longitudinal embolic member, wherein portions of the first and second longitudinal sections are configured in parallel within a lumen (before insertion in an aneurysm) and wherein these portions move apart from each other after exiting the lumen. In an example, this device can further comprise flexible embolic members which are substantially parallel as they travel through a longitudinal lumen and which separate from each other after they exit longitudinal lumen within an aneurysm sac. In an example, this device can further comprise a longitudinal member with shape memory.

In an example, a device for occluding an aneurysm can comprise: a plurality of connected longitudinal segments which is inserted into an aneurysm sac; wherein a longitudinal segment further comprises a first longitudinal wire comprising one (e.g. the right) side of the longitudinal segment, a second longitudinal wire comprising the opposite (e.g. the left) side of the longitudinal segment, and an inner mesh section spanning between the first longitudinal wire and the second longitudinal wire; wherein a longitudinal segment has a first configuration prior to insertion into the aneurysm sac wherein the first and second wires are a first average distance apart from each other and a second configuration after insertion into aneurysm sac wherein the first and second wires are a second average distance apart from each other; and wherein the second average distance is greater than the first average distance.

In an example, a device for occluding an aneurysm can comprise: a plurality of connected longitudinal segments which is inserted into an aneurysm sac; wherein each longitudinal segment further comprises a first longitudinal wire comprising one (e.g. the right) side of the longitudinal segment, a second longitudinal wire comprising the opposite (e.g. the left) side of the longitudinal segment, and an inner mesh section spanning between the first longitudinal wire and the second longitudinal wire; wherein each longitudinal segment has a first configuration prior to insertion into the aneurysm sac wherein the first and second wires are a first average distance apart from each other and a second configuration after insertion into aneurysm sac wherein the first and second wires are a second average distance apart from each other; and wherein the second average distance is greater than the first average distance.

In an example, a longitudinal section can look similar to a flower petal. In an example, a longitudinal section can have a shape selected from the group consisting of: arcuate section of the surface of a sphere (such as a longitudinal slice area of a globe), circle, conic section, convex lens, crescent, cylindrical section, ellipse with central longitudinal section removed and the remaining two sides connected, flame shape, flower petal, full ellipse, half circle, helix, hourglass, hyperbola, keystone, leaf, lemon shape, one phase (positive or negative) of a sinusoidal wave, onion-shape, orange segment, oval, pear shape, river (area between two parallel in-phase sine waves), rounded rectangle, “s”-shape, spherical section, spiral, tear drop, torus, and yin or yang portion of a yin/yang symbol.

In an example, a first longitudinal wire can form (be part of) one side perimeter (e.g. the right side) of a longitudinal segment and a second longitudinal wire can form (be part of) the other side perimeter (e.g. the left side) of the longitudinal segment. In an example, a first or second longitudinal wire (coil, strand, or fiber) can be arcuate. In an example, a first or second longitudinal wire (coil, strand, or fiber) can be undulating, wavy, sinusoidal, cataracted, and/or scalloped shape. In an example, a first or second longitudinal wire (coil, strand, or fiber) can be straight or have a zigzag shape.

In an example, a multiple-width longitudinal mesh can comprise an alternating sequence of contiguous longitudinal segments—alternating between a narrow longitudinal segment and a wide longitudinal segment. In an example, two wide longitudinal segments which are connected by a narrow longitudinal segment can be called “connected.” In an example, a longitudinal wire (or coil, strand, or fiber) can form the right-side perimeter of one segment in a pair of connected wide longitudinal segments and the same wire can also form the right-side perimeter of the other segment in that pair. Alternatively, a first longitudinal wire and a second longitudinal wire can cross and/or intersect between connected wide longitudinal segments. In the latter example, a longitudinal wire can form the right-side perimeter of one segment in a pair of connected wide longitudinal segments and the same wire can form the left-side perimeter of the other segment in that pair.

In an example, a plurality of wide longitudinal segments can each have the same shape and/or size. In an example, wide longitudinal segments within a plurality can have different shapes and/or sizes. In an example, successive loops and/or wide longitudinal segments can become smaller to better fill the central space of an aneurysm sac. In an example, wide longitudinal segments can be arranged in distal-to-proximal sequence of decreasing size, especially if they are configured to form a sphere by accumulation of mass in an “outside-to-inside” manner within a sphere. In an example, a distal (outer-placed) segment in distal-to-proximal sequence of wide longitudinal segments can be larger than a proximal (inner-placed) segment in that sequence. In an example, longitudinal segments can be arranged in distal-to-proximal sequence of increasing size, especially if they are configured to form a sphere by accumulation of mass in an “inside-to-outside” manner around the sphere. In an example, a distal (inner-placed) segment in distal-to-proximal sequence of wide longitudinal segments can be smaller than a proximal (outer-placed) segment in that sequence.

In an example, this device can further comprise a connector which connects a first longitudinal wire to a second longitudinal wire. In an example, this device can further comprise a connector which connects a first longitudinal wire to a second longitudinal wire within a narrow longitudinal section. In an example, a connector can be a ring or band which holds a first longitudinal wire and a second longitudinal wire together. In an example, a connector can include a moveable joint, axle, or hinge which allows a first longitudinal wire and a second longitudinal wire to move relative to each other. In an example, a connector can include a moveable joint, axle, or hinge which allows changes in the intersection angle between a first longitudinal wire and a second longitudinal wire. In an example, a connector can include a moveable joint, axle, or hinge which allows the intersection angle between a first longitudinal wire and a second longitudinal wire to change between a first device configuration (before insertion) and a second device configuration (after insertion).

In an example, a first longitudinal wire can have an undulating, wavy, sinusoidal, cataracted, and/or scalloped shape and a second longitudinal wire can also have an undulating, wavy, sinusoidal, cataracted, and/or scalloped shape that is parallel to that of the first longitudinal wire. In an example, a first longitudinal wire can have an undulating, wavy, sinusoidal, cataracted, and/or scalloped shape and a second longitudinal wire can have an undulating, wavy, sinusoidal, cataracted, and/or scalloped shape that is symmetric to that of the first longitudinal wire (e.g. reflected relative to a central longitudinal axis of the multiple-width longitudinal mesh). In an example, a first longitudinal wire can have an undulating, wavy, sinusoidal, cataracted, and/or scalloped shape and a second longitudinal wire can have an undulating, wavy, sinusoidal, cataracted, and/or scalloped shape that is symmetric to that of the first longitudinal wire (e.g. reflected relative to a central longitudinal axis of the first longitudinal wire).

In an example, a first longitudinal wire can have an undulating, wavy, sinusoidal, cataracted, and/or scalloped shape and a second longitudinal wire can be straight. In an example, when a first longitudinal wire is arcuate and a second longitudinal wire is straight, then wide longitudinal sections can have scalloped, half-circle, or single phase (positive or negative) of a sinusoidal wave shapes. In an example, when a first longitudinal wire is arcuate and a second longitudinal wire is straight, then a sequence of wide longitudinal sections can have single-phase (positive or negative) sinusoidal wave shapes in alternating (e.g. right or left facing) directions.

In an example, a longitudinal section can look similar to a flower petal. In an example, a wide longitudinal section can have a shape selected from the group consisting of: arcuate section of the surface of a sphere (such as a longitudinal slice area of a globe), circle, conic section, convex lens, crescent, cylindrical section, ellipse with central longitudinal section removed and remaining two sides connected, flame shape, flower petal, full ellipse, half circle, helix, hourglass, hyperbola, keystone, leaf, lemon shape, one phase (positive or negative) of a sinusoidal wave, onion-shape, orange segment, oval, pear shape, river (area between two parallel in-phase sine waves), rounded rectangle, “s”-shape, spherical section, spiral, tear drop, torus, and yin or yang portion of a yin/yang symbol.

In an example, an inner mesh section between first and second longitudinal wires can have a shape selected from the group consisting of: arcuate section of the surface of a sphere (such as a longitudinal slice area of a globe), circle, conic section, convex lens, crescent, cylindrical section, ellipse with central longitudinal section removed and remaining two sides connected, flame shape, flower petal, full ellipse, half circle, helix, hourglass, hyperbola, keystone, leaf, lemon shape, one phase (positive or negative) of a sinusoidal wave, onion-shape, orange segment, oval, pear shape, river (area between two parallel in-phase sine waves), rounded rectangle, “s”-shape, spherical section, spiral, tear drop, torus, and yin or yang portion of a yin/yang symbol.

In an example, a mesh area within a wide longitudinal section whose right and left perimeters are defined by first and second longitudinal wires can have a shape selected from the group consisting of: arcuate section of the surface of a sphere (such as a longitudinal slice area of a globe), circle, conic section, convex lens, crescent, cylindrical section, ellipse with central longitudinal section removed and remaining two sides connected, flame shape, flower petal, full ellipse, half circle, helix, hourglass, hyperbola, keystone, leaf, lemon shape, one phase (positive or negative) of a sinusoidal wave, onion-shape, orange segment, oval, pear shape, river (area between two parallel in-phase sine waves), rounded rectangle, “s”-shape, spherical section, spiral, tear drop, torus, and yin or yang portion of a yin/yang symbol.

In an example: a longitudinal wire (or coil, strand, or fiber) can have a first level of flexibility, bendability, stretchabilty, or elasticity; an inner mesh section (or net or low-porosity barrier) can have a second level of flexibility, bendability, stretchability, or elasticity; and the second level can be greater than the first level. In an example: a longitudinal wire can have a first level of stiffness, resilience, or tensile strength; an inner mesh section can have a second level of stiffness, resilience, or tensile strength; and the second level can be less than the first level. In an example: a longitudinal wire can have a first diameter or thickness; an inner mesh section can have a second diameter or thickness; and the second diameter or thickness can be less than the first second diameter or thickness.

In an example a longitudinal wire can be made from metal and an inner mesh section can be made from a polymer. In an example a longitudinal wire and an inner mesh section can both be made from metal. In an example, an inner mesh section can be braided or woven. In an example, an inner mesh section can be relatively thin. In an example, the thickness of an inner mesh section can be less than 25% of the width of an inner mesh section. In an example, the thickness of an inner mesh section can be less than 10% of the width of an inner mesh section. In an example, the thickness of an inner mesh section can be less than 1% of the width of an inner mesh section.

In an example: a first (e.g. right-side) longitudinal wire can have a first level of flexibility, resiliency, tensile strength, and/or elasticity; a second (e.g. left-side) longitudinal wire can have a second level of flexibility, resiliency, tensile strength, and/or elasticity; and the first level can be greater than the second level. In an example, having a difference in the level of flexibility, resiliency, tensile strength, and/or elasticity between first and second longitudinal wires can bias connected wide longitudinal sections to move toward each other in an asymmetric manner in order to form a sphere, ellipsoid, or other arcuate three-dimensional mass as they accumulate within the aneurysm sac. In an example, longitudinally-asymmetric (e.g. right vs. left side) flexibility, resiliency, tensile strength, and/or elasticity in first and second longitudinal wires can cause sequential wide longitudinal sections to curve around each other in order to form a sphere, ellipsoid, or other arcuate three-dimensional mass within an aneurysm sac. In an example, this device can comprise an elongated member with shape memory. In an example, the accumulation of curved, looping embolic members in aneurysm sac can create a looping embolic mass that substantively occludes the aneurysm sac.

In an example, a mesh need not be of uniform tensile strength, flexibility, plasticity, or elasticity. In an example, different regions of a mesh can have a different strengths, flexibilities, plasticities, or elasticities. In an example: a first (e.g. right side) portion of an inner mesh section can have a first level of flexibility, resiliency, tensile strength, and/or elasticity; a second (e.g. left side) portion of an inner mesh section can have a second level of flexibility, resiliency, tensile strength, and/or elasticity; and the first level can be greater than the second level. In an example, having a difference in the level of flexibility, resiliency, tensile strength, and/or elasticity between first and second portions of an inner mesh section can bias connected wide longitudinal sections to move toward each other in an asymmetric manner in order to form a sphere, ellipsoid, or other arcuate three-dimensional mass as they accumulate within the aneurysm sac. In an example, longitudinally-asymmetric (e.g. right vs. left side) flexibility, resiliency, tensile strength, and/or elasticity in first and second portions of an inner mesh section can cause sequential wide longitudinal sections to curve around each other in order to form a sphere, ellipsoid, or other arcuate three-dimensional mass within an aneurysm sac. In an example, accumulation of curved, looping embolic members in aneurysm sac can create a looping embolic mass that substantively occludes the aneurysm sac.

In an example, an inner mesh section can comprise a single layer of mesh, net, or fabric. In an example, an inner mesh section can comprise two or more layers of mesh, net, or fabric. In an example, a plurality of embolic members can be inserted between layers of an inner mesh section. In an example, a plurality of soft and compressible fill members can be pumped or otherwise inserted between layers of an inner mesh section. In an example, fill members can be selected from the group consisting of: sponge material, foam material, and gel material. In an example, an inner mesh section can comprise a woven mesh of metal wires, threads, or strands. In an example, an inner mesh section can comprise a woven mesh of polymer threads or strands. In an example, an inner mesh section can be made from a nylon material, a polypropylene material, a polyester material, a polytetrafluoroethylene material, a poglianochris material or a polytetrafluoroethene material. In an example, an inner mesh section can comprise a stretchable and/or elastic fabric.

In an example, an inner mesh section can span at least 50% of the area between a first longitudinal wire and a second longitudinal wire. In an example, an inner mesh section can span at least 75% of the area between a first longitudinal wire and a second longitudinal wire. In an example, an inner mesh section can span at least 90% of the area between a first longitudinal wire and a second longitudinal wire. In an example, an inner mesh section can span the entire area between a first longitudinal wire and a second longitudinal wire.

In an example, this device can comprise a non-porous or low-porosity barrier. In an example, this device can comprise a non-porous or low-porosity barrier with one or more layers. In an example, a barrier can be made from a polymer film or fabric. In an example, a barrier can be stretchable and/or elastic. In an example, a barrier can be folded, pleated, or rolled in a first configuration (before insertion into the aneurysm sac) and unfolded, unpleated, or unrolled in a second configuration (after insertion into the aneurysm sac. In an example, a mesh can be initially folded, compressed, and/or in a relatively-collapsed form while it is being intravascularly guided through the body prior to insertion into an aneurysm sac. In an example, one or more soft and compressible fill members can be inserted between two barrier layers. In an example, fill members can be selected from the group consisting of: sponge material, foam material, and gel material. In an example, a flowable substance (such as saline solution or gel) can be pumped into a compartment between two barrier layers.

In an example, a non-porous or low-porosity barrier can span at least 50% of the area between a first longitudinal wire and a second longitudinal wire. In an example, a barrier can span at least 75% of the area between a first longitudinal wire and a second longitudinal wire. In an example, a barrier can span at least 90% of the area between a first longitudinal wire and a second longitudinal wire. In an example, a barrier can span the entire area between a first longitudinal wire and a second longitudinal wire.

In an example, a device for occluding an aneurysm can comprise: a multiple-width longitudinal low-porosity ribbon which is configured to be inserted into an aneurysm sac; wherein the low-porosity ribbon has a first configuration prior to insertion into the aneurysm sac and a second configuration after insertion into aneurysm sac; wherein the low-porosity ribbon has a distal-to-proximal longitudinal axis in the first configuration; wherein the low-porosity ribbon has a length dimension along (or parallel to) the distal-to-proximal longitudinal axis, wherein the low-porosity ribbon has a width dimension perpendicular (or orthogonal) to the length dimension, and wherein the low-porosity ribbon has a thickness dimension perpendicular (or orthogonal) to the length dimension and the width dimension; wherein the low-porosity ribbon further comprises a plurality of narrow longitudinal segments with a first average length, a first average width, and a first average thickness; wherein the low-porosity ribbon further comprises a plurality of wide longitudinal segments with a second average length, a second average width, and a second average thickness; and wherein the second average width is at least twice the first average width in the second configuration; wherein the second average width is at least twice the second average thickness in the second configuration; and wherein the second average length is at least equal to the first average length in the second configuration.

In an example, a device for occluding an aneurysm can comprise: a low-porosity ribbon which further comprises a plurality of connected longitudinal segments which is inserted into an aneurysm sac; wherein each longitudinal segment further comprises a first longitudinal wire comprising a first (e.g. right) side of the longitudinal segment, a second longitudinal wire comprising the opposite (e.g. left) side of the longitudinal segment, and a flexible low-porosity barrier spanning between the first longitudinal wire and the second longitudinal wire; wherein each longitudinal segment has a first configuration prior to insertion into the aneurysm sac wherein the first and second wires are a first average distance apart from each other and a second configuration with a second width after insertion into aneurysm sac wherein the first and second wires are a second average distance apart from each other; and wherein the second average distance is greater than the first average distance.

In an example, narrow longitudinal segments and wide longitudinal segments can be contiguous with each other. In an example, a longitudinal section of a low-porosity ribbon device can have a shape selected from the group consisting of: arcuate section of the surface of a sphere (such as longitudinal sections of a globe), circle, conic section, convex lens, crescent, cylindrical section, ellipse with central longitudinal section removed and remaining two sides connected, flame shape, flower petal, full ellipse, half circle, helix, hourglass, hyperbola, keystone, leaf, lemon shape, one phase (positive or negative) of a sinusoidal wave, onion-shape, orange segment, oval, pear shape, river (area between two parallel in-phase sine waves), rounded rectangle, “s”-shape, spherical section, spiral, tear drop, torus, and yin or yang portion of yin/yang symbol.

In an example, two wide longitudinal sections of this device can be connected by one or more wires, cords, strings, springs, or bands. In an example, two wide longitudinal sections can be centrally connected by one or more wires, cords, strings, or bands. In an example, two wide longitudinal sections can be symmetrically connected by one or more wires, cords, strings, or bands. In an example, two wide longitudinal sections can be asymmetrically connected by two or more wires, cords, strings, or bands. In an example, two wide longitudinal sections can be tangentially connected by one or more wires, cords, strings, or bands.

In an example, two wide longitudinal sections can be connected by two or more elastic members with longitudinally-asymmetric (e.g. right vs. left side) elasticity which causes the two sections to move toward each other in an asymmetric (e.g. right vs. left) manner as they are inserted into an aneurysm sac. In an example, two wide longitudinal sections can be connected by a right-side elastic band with a first level of elasticity and by a left-side elastic band with a second level of elasticity, wherein the first level is different than the second level. In an example, asymmetric elasticity causes asymmetric movement which causes sequential wide longitudinal sections to curve around each other in order to form a sphere, ellipsoid, or other arcuate three-dimensional mass within an aneurysm sac.

In an example, two wide longitudinal sections can be connected by an elastic member which causes them to move toward each other after they are inserted into an aneurysm sac. In an example, two wide longitudinal sections can be asymmetrically connected by an elastic member (on their right or left side) which causes them to move asymmetrically toward each other after they are inserted into an aneurysm sac. In an example, this elastic-member-induced movement can cause the longitudinal sections to form a sphere, ellipse, or other arcuate three-dimensional shape.

In an example, two wide longitudinal sections can be connected by a pull-cord whose pulling by an operator causes them to move toward each other after they are inserted into an aneurysm sac. In an example, two wide longitudinal sections can be asymmetrically connected by a pull-cord, wherein pulling of this cord by an operator causes the two longitudinal sections to move asymmetrically toward each other after they are inserted into an aneurysm sac. In an example, this pull-cord-induced movement can cause the wide longitudinal sections to form a sphere, ellipse, or other arcuate three-dimensional shape.

In an example, the direction in which one longitudinal section of this device moves relative to another longitudinal section as the sections are inserted into an aneurysm can be steered, changed, and/or adjusted by a user in real time. In an example, the orientation of one longitudinal section relative to another longitudinal section can be steered and/or adjusted in real time as the sections are inserted into an aneurysm. In an example, the orientation of one longitudinal section relative to another longitudinal section can be steered, changed, and/or adjusted by a user during insertion of the sections into an aneurysm by one or more mechanisms selected from the group consisting of: selectively pulling on a string or cord which connects sections together; selectively connecting sections together (e.g. by fusing or crimping); selectively disconnecting sections (e.g. by cutting or melting connectors); selectively adjusting the tension and/or elasticity of connectors between sections; and selectively inflating balloons or other compartments between sections.

In an example, there can be variation in the lateral-location of narrow longitudinal sections which connect wide longitudinal sections. In an example, a first narrow longitudinal section connecting a first pair of wide longitudinal sections can be located to one side (e.g. to the right) of a central longitudinal axis of a multi-width longitudinal mesh and a second narrow longitudinal section connection a second pair of wide longitudinal sections can be located to the other side (e.g. to the left) of the central longitudinal axis. In an example, such asymmetric connection can cause sequential wide longitudinal sections to curve around each other in order to form a sphere, ellipsoid, or other arcuate three-dimensional mass within an aneurysm sac.

In an example, pairs of longitudinal sections can be connected to each other at different angles (relative to a longitudinal axis of a device). In an example, a distal-to-proximal sequence of longitudinal sections can be connected to each other at a progressive sequence of angles. In an example, a distal-to-proximal sequence of longitudinal sections can be connected to each other with distal-to-proximally-increasing angle degrees so that the longitudinal sections are biased to form a sphere, ellipsoid, or other three-dimensional mass upon insertion into an aneurysm sac. In an example, a distal-to-proximal sequence of longitudinal sections can be connected to each other with distal-to-proximally-decreasing angle degrees so that the longitudinal sections are biased to form a sphere, ellipsoid, or other three-dimensional mass upon insertion into an aneurysm sac. In an example, a distal-to-proximal sequence of longitudinal sections can be connected to each other with a distal-to-proximal sequence of alternating (greater, then lower) angle degrees so that the longitudinal sections are biased to form a spherical, elliptical, or other arcuate mass upon insertion into an aneurysm sac.

In an example, a device for occluding an aneurysm can comprise: a longitudinal lumen that is configured to be inserted into a blood vessel, wherein this blood vessel is the parent vessel from which an aneurysm has formed; a first flexible longitudinal embolic member that is configured to travel through the longitudinal lumen and be inserted into the aneurysm sac; a second flexible longitudinal embolic member that is configured to travel through the longitudinal lumen and be inserted into the aneurysm sac; wherein the longitudinal axes of the first and second flexible longitudinal embolic members are substantially parallel as these embolic members travel through the longitudinal lumen; and a plurality of connections which connect the first and second embolic members at a plurality of locations along their lengths; wherein segments of the first and second flexible longitudinal embolic members that are not connected by the connections move away from each other after they exit the longitudinal lumen, thereby forming loops within the aneurysm sac; wherein these loops are connected by the connections; and wherein accumulation of these loops within the aneurysm sac substantially occludes the aneurysm.

In an example, a longitudinal lumen can be a catheter. In an example, flexible longitudinal embolic members can be coils. In an example, connections can connect flexible longitudinal embolic members at uniformly-spaced locations along their lengths. In an example, connections which connect flexible longitudinal embolic members can be at non-uniform distances along their lengths in order to better occlude an aneurysm sac.

In an example, a device for occluding an aneurysm can comprise: a longitudinal lumen that is configured to be inserted into a blood vessel, wherein this blood vessel is the parent vessel from which an aneurysm has formed; a first flexible longitudinal embolic member that is configured to travel through the longitudinal lumen and be inserted into the aneurysm sac; a second flexible longitudinal embolic member that is configured to travel through the longitudinal lumen and be inserted into the aneurysm sac; wherein the longitudinal axes of the first and second embolic members are substantially parallel as these embolic members travel through the longitudinal lumen; a stretchable mesh which spans between the first flexible longitudinal embolic member and the second flexible longitudinal embolic member; and a plurality of connections which connect the first and second embolic members at a plurality of locations along the lengths of the embolic members; wherein the segments of the first and second embolic members that are not connected by the connections move away from each other after they exit the longitudinal lumen, thereby forming loops within the aneurysm sac; wherein these loops are connected by the connections; and wherein accumulation of these loops within the aneurysm sac substantially occludes the aneurysm.

In an example, a longitudinal lumen can be a catheter. In an example, flexible longitudinal embolic members can be coils. In an example, connections can connect flexible longitudinal embolic members at uniformly-spaced locations along their lengths. In an example, a stretchable mesh can be an elastic mesh. In an example, a stretchable mesh can be impermeable to blood flow. In an example, a stretchable mesh can resist blood flow. In an example, a stretchable mesh can fill the entire interior of a loop. In an example, a stretchable mesh can fill at least 50% of the interior of a loop.

In an example, this invention can be embodied in a device for occluding a cerebral aneurysm comprising: a longitudinal lumen that is configured to be inserted into a blood vessel; a first segment of a longitudinal embolic coil; a second segment of a longitudinal embolic coil, wherein the first and second segments are connected to each other at a proximal location along their length and are connected to each other at a distal location along their length, wherein the first and second segments have a first configuration when they are within the longitudinal lumen, wherein there is a first average distance between the first and second segments when they are in the first configuration, wherein the first and second segments have a second configuration after they exit the longitudinal lumen into an aneurysm sac, wherein there is a second average distance between the first and second segments when they are in the second configuration, and wherein the second distance is greater than the first distance; and a stretchable mesh which spans between the first and second segments.

In an example, the first and second segments can form a loop. In an example, this loop can be more circular in the second configuration than in the first configuration. In an example, this loop can have a greater interior area in the second configuration than in the first configuration. In an example, the stretchable mesh can fill the entire interior of a loop. In an example, the stretchable mesh can fill at least 50% of the interior of a loop. In an example, the stretchable mesh can resist blood flow.

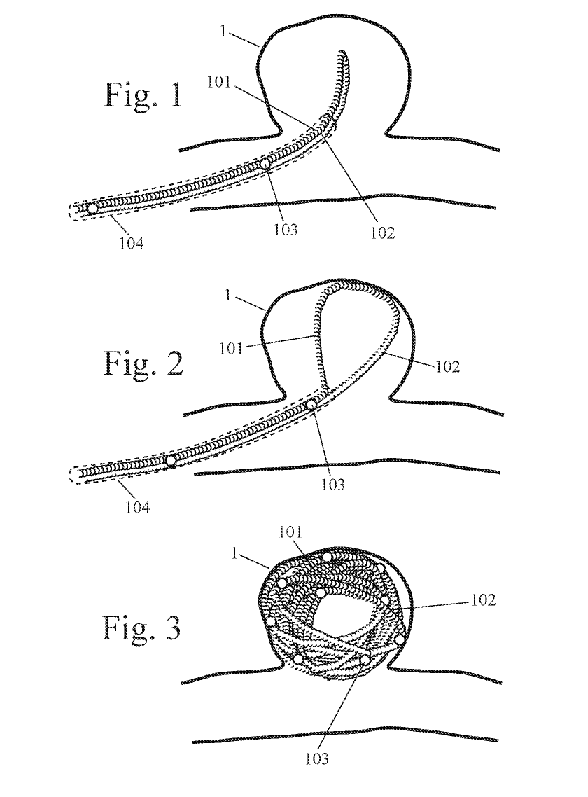

FIGS. 1 through 58 show some specific examples of how this invention can be embodied in an aneurysm occlusion device, but do not restrict the full generalizability of the final claims. Example and component variations which have been discussed thus far in this disclosure (and also in other disclosures which are linked by priority claim) can be applied where relevant to the examples in FIGS. 1 through 58 but are not repeated in the narratives accompanying these figures in order to reduce duplicative content.

We now discuss FIGS. 1 through 58 in detail. FIGS. 1 through 3 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm; (c) a plurality of connections between the first and second longitudinal sections, wherein these connections connect the first and second longitudinal sections at a plurality of selected locations along their longitudinal axes; and (d) a longitudinal lumen that is configured to be inserted into a blood vessel, wherein the first and second longitudinal sections travel through the lumen in order to be inserted into the aneurysm; wherein at least portions of the first and second longitudinal sections are configured in parallel within the lumen; wherein portions of the first and second longitudinal sections which are not connected by connections move apart from each other after exiting the lumen and the connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; and wherein loops are interconnected at the connections.

FIGS. 1 through 3 also show an example of a device to occlude an aneurysm comprising: (a) a longitudinal lumen that is configured to be inserted into a blood vessel, wherein this blood vessel is the parent vessel from which an aneurysm has formed; (b) a first flexible longitudinal embolic member that is configured to travel through the longitudinal lumen and be inserted into the aneurysm sac; (c) a second flexible longitudinal embolic member that is configured to travel through the longitudinal lumen and be inserted into the aneurysm sac; wherein the longitudinal axes of the first and second flexible longitudinal embolic members are substantially parallel as these flexible longitudinal embolic members travel through the longitudinal lumen; and (d) a plurality of connections which connect the first and second flexible longitudinal embolic members at a plurality of locations along the lengths of the flexible longitudinal embolic members; wherein the segments of the first and second flexible longitudinal embolic members that are not connected by the connections move away from each other after they exit the longitudinal lumen, thereby forming loops within the aneurysm sac; wherein these loops are connected by the connections; and wherein accumulation of these loops within the aneurysm sac substantially occludes the aneurysm.

In an example, a longitudinal lumen can be a removable catheter. In an example, flexible longitudinal embolic members can be coils. In an example, connections can connect flexible longitudinal embolic members at uniformly-spaced locations along their lengths so as to form equal-size loops within the aneurysm sac and wherein these equal-size loops substantially span the circumference of the aneurysm sac. In an example, connections can connect flexible longitudinal embolic members at uniformly-spaced locations along their lengths so as to form equal-size loops within the aneurysm sac and these equal-size loops substantially span the circumference of the aneurysm sac without protruding into the parent vessel. In an example, connections can connect flexible longitudinal embolic members at non-uniformly-spaced locations along their lengths so as to form loops of different sizes within the aneurysm sac and these different size loops substantially occlude the interior as well as the circumference of the aneurysm sac.

We now discuss the specific components of FIGS. 1 through 3 in detail. FIGS. 1 through 3 show three sequential views of the same example of a device and method to occlude an aneurysm. To provide anatomical context, FIG. 1 shows a longitudinal cross-sectional view of an aneurysm sac 1 which has formed on a longitudinal blood vessel. FIG. 1 also shows an occlusive device comprising: a longitudinal lumen 104 that has been inserted into the longitudinal blood vessel; a first flexible longitudinal embolic member 101 that travels through lumen 104 into aneurysm sac 1; a second flexible longitudinal embolic member 102 that travels through lumen 104 into aneurysm sac 1; and a plurality of connections (including 103) which connect first and second embolic members 101 and 102 at a plurality of locations along their longitudinal lengths. In this example, flexible longitudinal embolic members 101 and 102 are two different segments (or sides) of the same continuous flexible longitudinal embolic member. In this example, this continuous member has two parallel segments or sides (comprising flexible longitudinal embolic members 101 and 102) within longitudinal lumen 104. In another example, embolic member 101 and embolic 102 can be different embolic members that are connected in some other manner at their distal ends.

In this example, flexible longitudinal embolic members 101 and 102 are substantially parallel as they travel through longitudinal lumen 104. However, as shown in FIG. 2, portions of embolic members 101 and 102 which are not connected to each other separate from each other after they exit longitudinal lumen 104 within aneurysm sac 1. In an example, this separation can be partly caused by pressure from contact with the wall of aneurysm sac 1. In an example, this separation can be partly caused by embolic members 101 and 102 having a shape memory with a shape that is restored after these embolic members exit longitudinal lumen 104. In the example that is shown in FIGS. 1 and 2, segments of embolic members 101 and 102 which are not connected by connections (such as 103) move away from each other after they exit longitudinal lumen 104.

FIG. 3 shows the accumulation of a plurality of interconnected, contiguous loops within aneurysm sac 1 as flexible longitudinal embolic members 101 and 102 continue to be pushed into aneurysm sac 1. These loops are pair-wise connected to each other by the plurality of connections (including connection 103). As shown in FIG. 3, accumulation of this plurality of loops within aneurysm sac 1 forms an embolic mass which substantially occludes the aneurysm. In this example, the interconnected and contiguous nature of these loops helps to prevent loops from prolapsing out of aneurysm sac 1 into the parent blood vessel. This can result in less prolapse of coils into the parent vessel than is the case with coils in the prior art which disperse and accumulate in a free-form spiraling manner within the aneurysm sac. Also, FIG. 3 shows longitudinal lumen 104 as having been removed.

In the example shown in FIGS. 1 through 3, the connections (such as 103) between embolic members 101 and 102 are relatively evenly-spaced along the longitudinal lengths of embolic members 101 and 102. In an example, the spacing of these connections can be selected for a specific aneurysm with a specific size and shape in order to most efficiently occlude that specific aneurysm. In an example, the spacing of connections can differ between devices which are configured to occlude narrow-neck aneurysms and devices which are configured to occlude wide-neck aneurysms. In an example, the spacing of these connections can be pre-selected to vary along the length of embolic members 101 and 102 in order to most efficiently occlude an aneurysm at different times or stages during the occlusion procedure. For example, connections can be separated by longer distances at the most distal portions of embolic members 101 and 102 and become progressively shorter at more proximal portions of embolic members 101 and 102. In an example, the most distal connections can be spaced to form loops which span the entire circumference of the aneurysm sac but successive loops can become smaller and smaller to better fill the central space of the aneurysm sac. Relevant design variations discussed elsewhere in this disclosure or in priority-linked disclosures can also be applied to the example shown here.

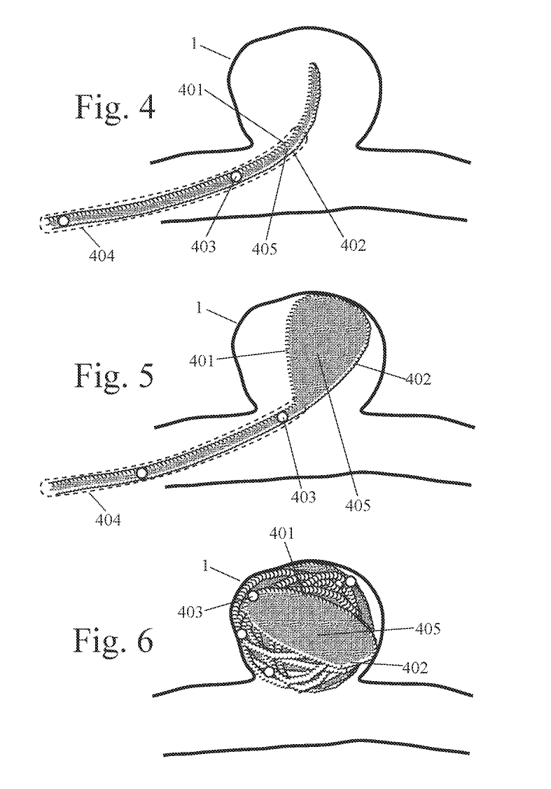

FIGS. 4 through 6 show another example of a device and method to occlude an aneurysm which is like the example shown in FIGS. 1 through 3, except that there is also a stretchable mesh within the loops. FIGS. 4 through 6 show three sequential views of a device and method to occlude an aneurysm which can be described as embolic coils which form interconnected contiguous loops within an aneurysm sac, wherein the interiors of these loops are spanned by a stretchable mesh.

More specifically, FIGS. 4 through 6 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm; (c) a stretchable mesh which spans between the first and second longitudinal sections; (d) a plurality of connections between the first and second longitudinal sections, wherein these connections connect the first and second longitudinal sections at a plurality of selected locations along their longitudinal axes; and (e) a longitudinal lumen that is configured to be inserted into a blood vessel; wherein the first and second longitudinal sections travel through the lumen in order to be inserted into the aneurysm; wherein at least portions of the first and second longitudinal sections are configured in parallel within the lumen; wherein portions of the first and second longitudinal sections which are not connected by connections move apart from each other after exiting the lumen and connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; and wherein loops are interconnected at the connections.

FIGS. 4 through 6 also show an example of a device to occlude an aneurysm comprising: (a) a flexible longitudinal lumen that is configured to be inserted into a blood vessel, wherein this blood vessel is the parent vessel from which an aneurysm has formed; (b) a first flexible longitudinal embolic member that is configured to travel through the flexible longitudinal lumen and be inserted into the aneurysm sac; (c) a second flexible longitudinal embolic member that is configured to travel through the flexible longitudinal lumen and be inserted into the aneurysm sac; wherein the flexible longitudinal axes of the first and second longitudinal embolic members are substantially parallel as these longitudinal embolic members travel through the flexible longitudinal lumen; (d) a stretchable mesh which spans between the first and second flexible longitudinal sections; and (e) a plurality of connections which connect the first and second longitudinal embolic members at a plurality of locations along the lengths of the longitudinal embolic members; wherein the segments of the first and second longitudinal embolic members that are not connected by the connections move away from each other after they exit the flexible longitudinal lumen, thereby forming loops within the aneurysm sac; wherein these loops are connected by the connections; and wherein accumulation of these loops and the stretchable mesh within the aneurysm sac substantially occludes the aneurysm.

In an example, a longitudinal lumen can be a removable catheter. In an example, flexible longitudinal embolic members can be coils. In an example, connections can connect flexible longitudinal embolic members at uniformly-spaced locations along their lengths so as to form equal-size loops within the aneurysm sac and wherein these equal-size loops substantially span the circumference of the aneurysm sac. In an example, connections can connect flexible longitudinal embolic members at uniformly-spaced locations along their lengths so as to form equal-size loops within the aneurysm sac and these equal-size loops substantially span the circumference of the aneurysm sac without protruding into the parent vessel. In an example, connections can connect flexible longitudinal embolic members at non-uniformly-spaced locations along their lengths so as to form loops of different sizes within the aneurysm sac and these different size loops substantially occlude the interior as well as the circumference of the aneurysm sac. In an example, the embolic members can criss-cross each other at their connections, wherein the embolic members switch sides from one loop to the next. In an example, sinusoidal embolic members can criss-cross each other at their connections, wherein the embolic members switch sides from one loop to the next.

In an example, a stretchable mesh can be an elastic mesh. In an example, a stretchable mesh can be made from a polymer. In an example, a stretchable mesh can be made from metal. In an example, a stretchable mesh can be attached to the first and second flexible longitudinal embolic members. In an example, a stretchable mesh can loop around the first and second flexible longitudinal embolic members. In an example, a stretchable mesh can span the entire interiors of loops. In an example, a stretchable mesh can span at least 50% of the interiors of loops. In an example, a stretchable mesh can be impermeable to blood flow. In an example, a stretchable mesh can resist blood flow.

We now discuss the specific components of FIGS. 4 through 6 in detail. FIGS. 4 through 6 show three sequential views of the same example of a device and method to occlude an aneurysm. To provide anatomical context, FIG. 4 shows a longitudinal cross-sectional view of an aneurysm sac 1 which has formed on a longitudinal blood vessel. FIG. 4 also shows an occlusive device comprising: a longitudinal lumen 404 that has been inserted into the longitudinal blood vessel; a first flexible longitudinal embolic member 401 that travels through lumen 404 into aneurysm sac 1; a second flexible longitudinal embolic member 402 that travels through lumen 404 into aneurysm sac 1; a stretchable mesh 405 which spans between first flexible longitudinal embolic member 401 and second flexible longitudinal embolic member 402; and a plurality of connections (including 403) which connect first and second flexible longitudinal embolic members 401 and 402 at a plurality of locations along their longitudinal lengths. In this example, flexible longitudinal embolic members 401 and 402 are two different segments (or sides) of the same continuous embolic member. In this example, this continuous embolic member has two parallel segments or sides (comprising flexible longitudinal embolic members 401 and 402) within longitudinal lumen 404. In another example, flexible longitudinal embolic member 401 and flexible longitudinal embolic member 402 can be different flexible longitudinal embolic members that are connected in some other manner at their distal ends.

In this example, flexible longitudinal embolic members 401 and 402 are substantially parallel as they travel through longitudinal lumen 404. However, as shown in FIG. 5, portions of flexible longitudinal embolic members 401 and 402 which are not connected to each other separate from each other after they exit longitudinal lumen 404 within aneurysm sac 1. In an example, this separation can be partly caused by pressure from contact with the wall of aneurysm sac 1. In an example, this separation can be partly caused by flexible longitudinal embolic members 401 and 402 having a shape memory with a shape that is restored after these embolic members exit longitudinal lumen 404. In the example that is shown in FIGS. 1 and 2, segments of flexible longitudinal embolic members 401 and 402 which are not connected by connections (such as 403) move away from each other after they exit longitudinal lumen 404.

FIG. 6 shows the accumulation of a plurality of interconnected, contiguous loops within aneurysm sac 1 as flexible longitudinal embolic members 401 and 402 continue to be pushed into aneurysm sac 1. These loops are pair-wise connected to each other by the plurality of connections (including connection 403). As shown in FIG. 6, the stretchable mesh stretches to span the arcuate interiors of these loops. As shown in FIG. 6, accumulation of this plurality of loops and the stretchable mesh within aneurysm sac 1 forms an embolic coil-and-mesh mass (such as a coil-and-mesh ball) which substantially occludes the aneurysm. In this example, the interconnected and contiguous nature of these loops helps to prevent loops from prolapsing out of aneurysm sac 1 into the parent blood vessel. This can result in less prolapse of coils into the parent vessel than is the case with coils in the prior art which disperse and accumulate in a free-form spiraling manner within the aneurysm sac. Also, FIG. 6 shows longitudinal lumen 404 as having been removed.

In the example shown in FIGS. 4 through 6, the connections (such as 403) between embolic members 401 and 402 are relatively evenly-spaced along the longitudinal lengths of embolic members 401 and 402. In an example, the spacing of these connections can be selected for a specific aneurysm with a specific size and shape in order to most efficiently occlude that specific aneurysm. In an example, the spacing of connections can differ between devices which are configured to occlude narrow-neck aneurysms and devices which are configured to occlude wide-neck aneurysms. In an example, the spacing of these connections can be pre-selected to vary along the length of embolic members 401 and 402 in order to most efficiently occlude an aneurysm at different times or stages during the occlusion procedure. For example, connections can be separated by longer distances at the most distal portions of embolic members 401 and 402 and become progressively shorter at more proximal portions of embolic members 401 and 402. In an example, the most distal connections can be spaced to form loops which span the entire circumference of the aneurysm sac but successive loops can become smaller and smaller to better fill the central space of the aneurysm sac. In an example, connections which connect flexible longitudinal embolic members can be at non-uniform distances along their lengths in order to better occlude an aneurysm sac.

In an example, the device shown in FIGS. 4 through 6 can be described as a device to occlude an aneurysm comprising a series of proximally-and-distally-connected mesh-filled loops. In an example, the device shown in FIGS. 4 through 6 can be described as a device to occlude an aneurysm comprising a series of proximally-and-distally-connected mesh-filled loops which overlap within an aneurysm sac to create a coil-and-mesh mass (such as a coil-and-mesh ball). In an example, the device shown in FIGS. 4 through 6 can be described as a device to occlude an aneurysm comprising a series of proximally-and-distally-connected mesh-filled loops whose sides are relatively parallel as they travel through a lumen and whose sides become concave after they exit the lumen into an aneurysm sac in order to create a coil-and-mesh mass (such as a coil-and-mesh ball).

In an example, FIGS. 4 though 6 show a device for occluding an aneurysm comprising: a catheter; a first segment of a longitudinal embolic coil; a second segment of a longitudinal embolic coil, wherein the first and second segments are connected to each other at a proximal location along their length and are connected to each other at a distal location along their length, wherein the first and second segments have a first configuration when they are within the catheter, wherein there is a first average distance between the first and second segments when they are in the first configuration, wherein the first and second segments have a second configuration after they exit the catheter into an aneurysm sac, wherein there is a second average distance between the first and second segments when they are in the second configuration, and wherein the second distance is greater than the first distance; and a stretchable mesh which spans between the first and second segments.

In an example, FIGS. 4 though 6 show a device for occluding an aneurysm comprising: a catheter; a first longitudinal embolic coil; a second longitudinal embolic coil, wherein the first and second longitudinal embolic coils are connected to each other at a plurality of locations along their lengths, forming a plurality of loops whose sides comprise segments of the first longitudinal embolic coil and segments of the second longitudinal embolic coil, wherein the first and second longitudinal embolic coils have a first configuration when they are within the catheter and a second configuration after they exit the catheter into an aneurysm sac, wherein the sides of the loops are further apart in the second configuration than in the first configuration; and a stretchable mesh which spans the interiors of the loops between the first and second longitudinal embolic coils. Relevant design variations discussed elsewhere in this disclosure or in priority-linked disclosures can also be applied to the example shown here.

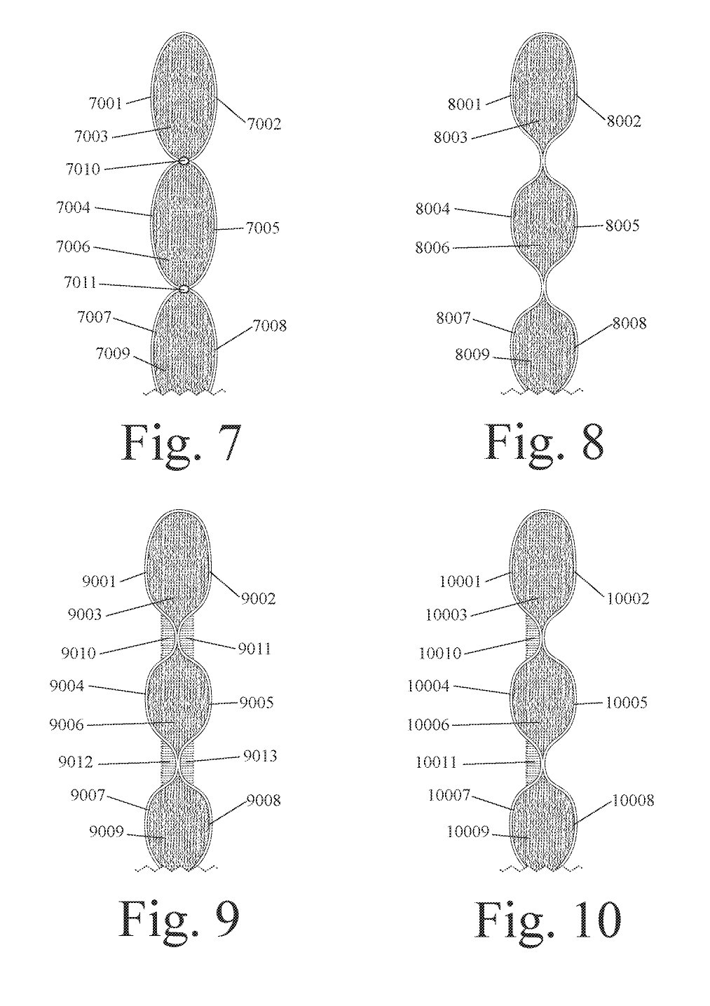

FIG. 7 shows an example of an aneurysm occlusion device with a plurality of connected longitudinal segments. Each longitudinal segment has a first longitudinal wire which forms one side of the segment, a second longitudinal wire which forms the opposite side of the segment, and an inner mesh which spans between the first and second wires. In this example, the first and second wires in a segment combine to form an oval or elliptical loop. This example also shows connectors between segments.

Specifically, FIG. 7 shows an aneurysm occlusion device with a plurality of longitudinal segments, three of which are shown here. Each of the three segments has a first longitudinal wire (7001, 7004, and 7007, respectively), a second longitudinal wire (7002, 7005, and 7008, respectively), and an inner mesh (7003, 7006, and 7009, respectively) which spans between the first and second wires. This example also includes connectors 7010 and 7011 between segments. Example variations and descriptions discussed elsewhere in this disclosure or in priority-linked disclosures can be applied to this example, but are not repeated here in order to reduce redundancy.

FIG. 8 shows an example of an aneurysm occlusion device with a plurality of connected longitudinal segments. Each longitudinal segment has a first longitudinal wire which forms one side of the segment, a second longitudinal wire which forms the opposite side of the segment, and an inner mesh which spans between the first and second wires. In this example, the same continuous first longitudinal wire spans the same side of each of the segments and the same continuous second longitudinal wire spans the opposite side of each of the segments. In this example, the longitudinal segments are shaped like flower petals.

Specifically, FIG. 8 shows an aneurysm occlusion device with a plurality of petal-shaped longitudinal segments, three of which are shown here. Each of the three segments has a first longitudinal wire (8001, 8004, and 8007, respectively), a second longitudinal wire (8002, 8005, and 8008, respectively), and an inner mesh (8003, 8006, and 8009, respectively) which spans between the first and second wires. In this example, the same continuous first longitudinal wire spans the same side of each of the three segments and the same continuous second longitudinal wire spans the opposite side of each of the three segments. In an example, a first longitudinal wire can span alternating (e.g. right vs. left) sides in a sequence of segments and a second longitudinal wire can span alternating (e.g. left vs. right) sides in the sequence of segments. In an example, different segments can have separate wires. Example variations and descriptions discussed elsewhere in this disclosure or in priority-linked disclosures can be applied to this example, but are not repeated here in order to reduce redundancy.

FIG. 9 shows an example of an aneurysm occlusion device with a plurality of connected longitudinal segments. Each longitudinal segment has a first longitudinal wire which forms one side of the segment, a second longitudinal wire which forms the opposite side of the segment, and a mesh which spans between the first and second wires. In this example, the same continuous first longitudinal wire spans the same side of each of the segments and the same continuous second longitudinal wire spans the opposite side of each of the segments. In this example, the longitudinal segments are shaped like flower petals. This example also includes left-side and right-side elastic connectors between segments.

Specifically, FIG. 9 shows an aneurysm occlusion device with a plurality of petal-shaped longitudinal segments, three of which are shown here. Each of the three segments has a first longitudinal wire (9001, 9004, and 9007, respectively), a second longitudinal wire (9002, 9005, and 9008, respectively), and an inner mesh (9003, 9006, and 9009, respectively) which spans between the first and second wires. This example also includes left-side and right-side elastic connectors (9010, 9011, 9012, and 9013) between segments. In an example, a first side connector (such as 9010) can have a first level of elasticity or flexibility, an opposite side connector (such as 9011) can have a second level of elasticity flexibility, and the first level can be different than the second level. In an example, differences in elasticity or flexibility between (left-side vs. right-side) connectors can bias the longitudinal axis of connected segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 10 shows an example of an aneurysm occlusion device that is similar to the one shown in FIG. 9 except that it has longitudinally asymmetric (one side only) elastic connectors between segments. Specifically, FIG. 10 shows an aneurysm occlusion device with a plurality of petal-shaped longitudinal segments, three of which are shown here. Each of the three segments has a first longitudinal wire (10001, 10004, and 10007, respectively), a second longitudinal wire (10002, 10005, and 10008, respectively), and an inner mesh (10003, 10006, and 10009, respectively) which spans between the first and second wires. This example also includes left-side-only elastic connectors (10010 and 10011) between segments. In an example, such longitudinally-asymmetric (e.g. left-side only) connectors can bias the longitudinal axis of connected segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

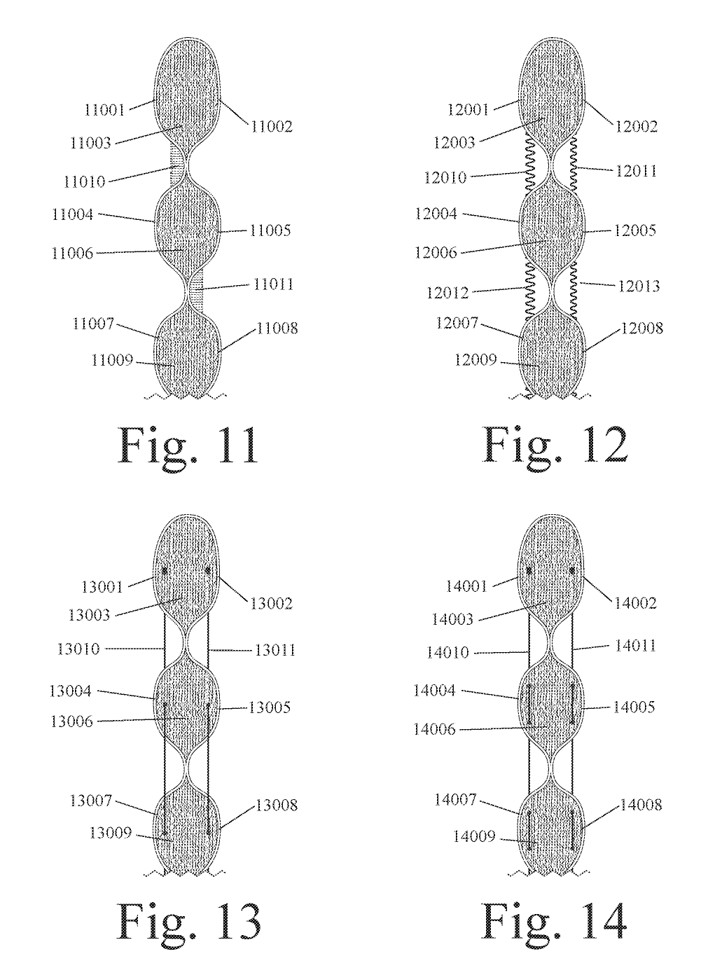

FIG. 11 shows an example of an aneurysm occlusion device that is similar to the one shown in FIG. 9 except that it has longitudinally asymmetric (alternating side) elastic connectors between segments. Specifically, FIG. 11 shows an aneurysm occlusion device with a plurality of petal-shaped longitudinal segments, three of which are shown here. Each of the three segments has a first longitudinal wire (11001, 11004, and 11007, respectively), a second longitudinal wire (11002, 11005, and 11008, respectively), and an inner mesh (11003, 11006, and 11009, respectively) which spans between the first and second wires. This example also includes alternating (e.g. left vs. right) side elastic connectors (11010 and 11011) between segments. In an example, such longitudinally-asymmetric (alternating side) connectors can bias the longitudinal axis of connected segments into curvature in a first direction and then in a second direction as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 12 shows an example of an aneurysm occlusion device with a plurality of connected petal-shaped longitudinal segments. Each longitudinal segment has a first longitudinal wire which forms one side of the segment, a second longitudinal wire which forms the opposite side of the segment, and a mesh which spans between the first and second wires. This example also includes left-side and right-side spring connectors between segments.

Specifically, FIG. 12 shows an aneurysm occlusion device with a plurality of petal-shaped longitudinal segments, three of which are shown here. Each of the three segments has a first longitudinal wire (12001, 12004, and 12007, respectively), a second longitudinal wire (12002, 12005, and 12008, respectively), and an inner mesh (12003, 12006, and 12009, respectively) which spans between the first and second wires. This example also includes left-side and right-side spring connectors (12010, 12011, 12012, and 12013) between segments. In an example, a first side spring (such as 12010) can have a first level of elasticity or tensile strength, an opposite side spring (such as 12011) can have a second level of elasticity or tensile strength, and the first level can be different than the second level. In an example, differences in elasticity or tensile strength between (left-side vs. right-side) springs can bias the longitudinal axis of connected segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 13 shows an example of an aneurysm occlusion device with a plurality of connected petal-shaped longitudinal segments. Each longitudinal segment has a first longitudinal wire which forms one side of the segment, a second longitudinal wire which forms the opposite side of the segment, and a mesh which spans between the first and second wires. This example also includes left-side and right-side pull-cords which span and connect segments.

Specifically, FIG. 13 shows an aneurysm occlusion device with a plurality of petal-shaped longitudinal segments, three of which are shown here. Each of the three segments has a first longitudinal wire (13001, 13004, and 13007, respectively), a second longitudinal wire (13002, 13005, and 13008, respectively), and an inner mesh (13003, 13006, and 13009, respectively) which spans between the first and second wires. This example also includes left-side and right-side pull-cords (13010 and 13011) which span and connect segments. In an example, when a user pulls on a pull-cord, it pulls longitudinal segments closer together. In this example, pulling on pull-cords draws segments together in an alternating side (e.g. zigzag) manner. In an example, differential pulling on left-side vs. right-side pull-cords can bias the longitudinal axis of connected segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 14 shows an example of an aneurysm occlusion device like the one in FIG. 13 except that pulling on pull-cords draws segments together in a same-side (spiral) manner. Specifically, FIG. 14 shows an aneurysm occlusion device with a plurality of petal-shaped longitudinal segments, three of which are shown here. Each of the three segments has a first longitudinal wire (14001, 14004, and 14007, respectively), a second longitudinal wire (14002, 14005, and 14008, respectively), and an inner mesh (14003, 14006, and 14009, respectively) which spans between the first and second wires. This example also includes left-side and right-side pull-cords (14010 and 14011) which span and connect segments. In this example, pulling on pull-cords draws segments together in a same-side (e.g. spiral) manner. In an example, differential pulling on left-side vs. right-side pull-cords can bias the longitudinal axis of connected segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

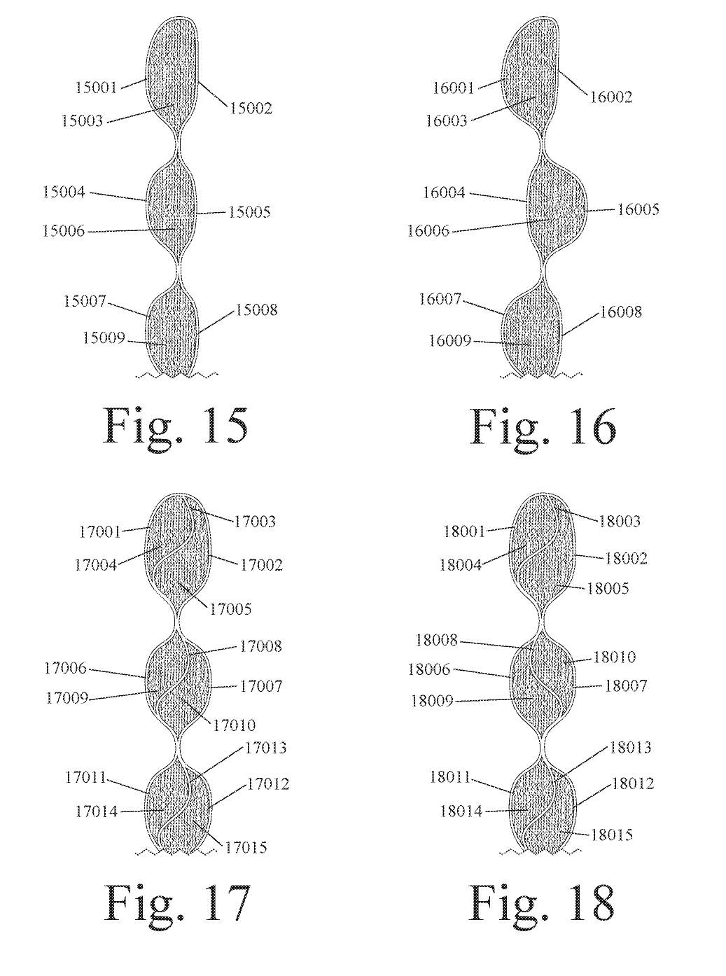

FIG. 15 shows an example of an aneurysm occlusion device with a plurality of connected longitudinal segments. Each longitudinal segment has a first longitudinal wire which forms one side of the segment, a second longitudinal wire which forms the opposite side of the segment, and a mesh which spans between the first and second wires. In this example, the longitudinal segments are geometrically-asymmetric with respect to their longitudinal axes, but each have the same shape and orientation.

Specifically, FIG. 15 shows an aneurysm occlusion device with a plurality of longitudinal segments which are geometrically-asymmetric with respect to their longitudinal axes. Three segments are shown here. Each of the three segments has a first longitudinal wire (15001, 15004, and 15007, respectively), a second longitudinal wire (15002, 15005, and 15008, respectively), and an inner mesh (15003, 15006, and 15009, respectively) which spans between the first and second wires. In this example, the three segments each have the same shape and orientation. The geometric-asymmetry of these segments with respect to their common longitudinal axis can bias this longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 16 shows an example of an aneurysm occlusion device with a plurality of connected longitudinal segments. Each longitudinal segment has a first longitudinal wire which forms one side of the segment, a second longitudinal wire which forms the opposite side of the segment, and a mesh which spans between the first and second wires. In this example, the longitudinal segments are geometrically-asymmetric with respect to their longitudinal axes. The segments each have the same shape, but differ in orientation.

Specifically, FIG. 16 shows an aneurysm occlusion device with a plurality of longitudinal segments which are geometrically-asymmetric with respect to their longitudinal axes. Three segments are shown here. Each of the three segments has a first longitudinal wire (16001, 16004, and 16007, respectively), a second longitudinal wire (16002, 16005, and 16008, respectively), and an inner mesh (16003, 16006, and 16009, respectively) which spans between the first and second wires. In this example, the three segments each have the same shape, but differ in orientation. The geometric-asymmetry of these segments with respect to their longitudinal axes and their differences in orientation can bias their common longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 17 shows an example of an aneurysm occlusion device with a plurality of connected petal-shaped longitudinal segments. Each longitudinal segment has a first longitudinal wire which forms one side of the segment, a second longitudinal wire which forms the opposite side of the segment, a mesh which spans between the first and second wires, and an undulating longitudinal wire between the first and second longitudinal wires.

Specifically, FIG. 17 shows an aneurysm occlusion device with a plurality of connected petal-shaped longitudinal segments. Three segments are shown here. Each of the three segments has a first longitudinal wire (17001, 17006, and 17011, respectively), a second longitudinal wire (17002, 17007, and 17012, respectively), a (two-part) mesh (17004 and 17005, 17009 and 17010, and 17014 and 17015, respectively) which spans between the first and second wires, and an undulating longitudinal wire (17003, 17008, and 17013, respectively) between the first and second wires. In an example, an undulating longitudinal wire can be sinusoidal. In an example, an undulating longitudinal wire can span a longitudinal segment in a distal-to-proximal manner. Undulating longitudinal wires can bias the common longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 18 shows an example of an aneurysm occlusion device like the one shown in FIG. 17 except that the orientation of undulating longitudinal wires differs between longitudinal segments. Specifically, FIG. 18 shows an aneurysm occlusion device with a plurality of connected petal-shaped longitudinal segments. Three segments are shown here. Each of the three segments has a first longitudinal wire (18001, 18006, and 18011, respectively), a second longitudinal wire (18002, 18007, and 18012, respectively), a (two-part) mesh (18004 and 18005, 18009 and 18010, and 18014 and 18015, respectively) which spans between the first and second wires, and an undulating longitudinal wire (18003, 18008, and 18013, respectively) between the first and second wires. Undulating longitudinal wires can bias the common longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

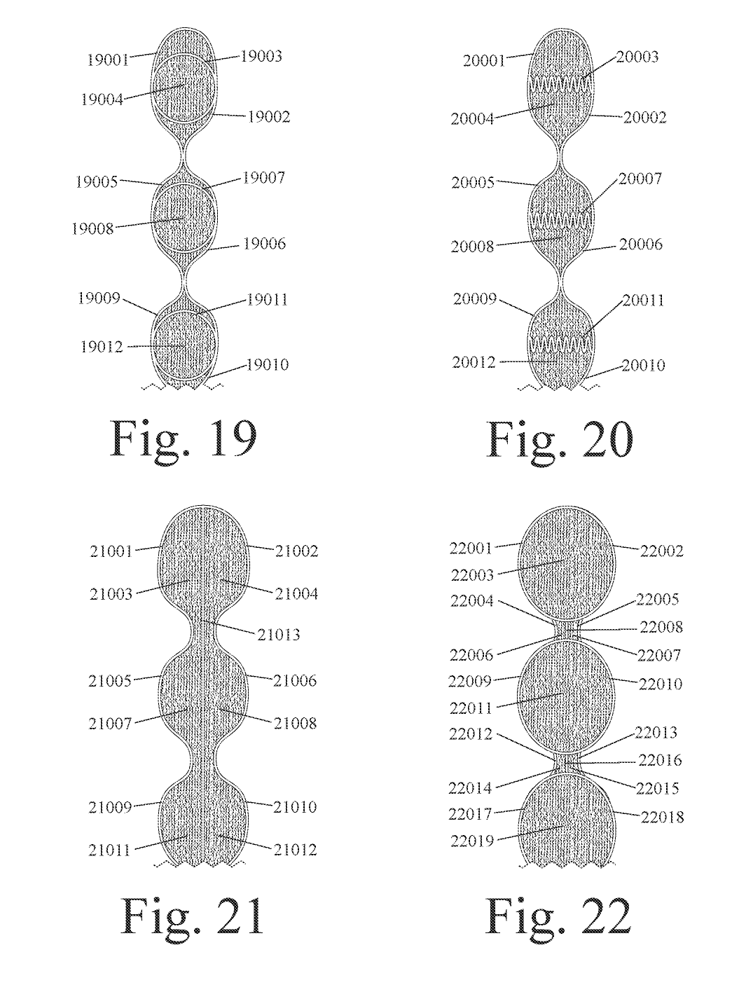

FIG. 19 shows an example of an aneurysm occlusion device with a plurality of connected petal-shaped longitudinal segments (three shown here), wherein each of the three segments has a first-side longitudinal wire (19001, 19005, and 19009, respectively), a second-side longitudinal wire (19002, 19006, and 19010, respectively), an inner wire loop (19003, 19007, and 19011, respectively) between the first-side and second-side wires, and a mesh (19004, 19008, and 19012, respectively) within the inner wire loop. In an example, an inner wire loop can be circular, elliptical, or oval. In an example, an inner wire loop can expand laterally and shrink longitudinally as a segment is inserted into an aneurysm sac. Example variations and descriptions discussed elsewhere in this disclosure or in priority-linked disclosures can be applied to this example, but are not repeated here in order to reduce redundancy.

FIG. 20 shows an example of an aneurysm occlusion device with a plurality of connected petal-shaped longitudinal segments (three shown here), wherein each of the three segments has a first-side longitudinal wire (20001, 20005, and 20009, respectively), a second-side longitudinal wire (20002, 20006, and 20010, respectively), a mesh (20004, 20008, and 20012, respectively) between the first-side and second-side wires, and a spring and/or coil (20003, 20007, and 20011, respectively) between the first-side and second-side wires. In an example, a spring and/or coil can connect first-side and second-side wires in a lateral manner. In an example, a spring and/or coil can push first-side and second-side wires apart as a segment is inserted into an aneurysm sac. Example variations and descriptions discussed elsewhere in this disclosure or in priority-linked disclosures can be applied to this example, but are not repeated here in order to reduce redundancy.

FIG. 21 shows an example of an aneurysm occlusion device with an undulating (e.g. sinusoidal-sided) sequence of (alternating) wide and narrow longitudinal segments (three shown here), wherein each of the three segments has a first-side longitudinal wire (21001, 21005, and 21009, respectively), a second-side longitudinal wire (21002, 21006, and 21010, respectively), and a two-part mesh (21003 and 21004, 21007 and 21008, and 21011 and 21012, respectively) between the first-side and second-side wires. This example also includes a central longitudinal wire 21013 which spans all segments. In this example, the same continuous first-side longitudinal wire also spans all segments and the same continuous second-side longitudinal wire also spans all segments. In other examples, there can be separate first-side and second-side side wires for different segments. In this example, the same side of a two-part mesh spans all segments. In other examples, there can be separate side meshes for different segments.

In an example, a first part of mesh (e.g. 21003) on a first side (e.g. left side) relative to a longitudinal axis can have a first level of elasticity and/or flexibility, a second part of mesh (e.g. 21004) on a second side (e.g. right side) of the longitudinal axis can have a second level of elasticity and/or flexibility, and the second level can be different than the first level. In an example, such a longitudinally-asymmetric (left-side vs. right-side) difference in mesh elasticity and/or flexibility can bias the longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 22 shows an example of an aneurysm occlusion device with a sequence of connected (alternating) wide and narrow longitudinal segments. Three wide segments and two narrow segments shown here. In this example, each of the three wide segments has a first-side wide segment wire (22001, 22009, and 22017, respectively), a second-side wide segment wire (22002, 22010, and 22018, respectively), and a wide segment mesh (22003, 22011, and 22019, respectively) between the first-side and second-side wide segment wires. In this example, the first-side wide segment wire and the second-side wide segment wire are both part of the same continuous circular, elliptical, or oval loop. In this example, each of the two narrow segments has a first-side narrow segment wire (22004 and 22012, respectively), a second-side narrow segment wire (22005 and 22013, respectively), a central longitudinal narrow segment wire (22008 and 22016, respectively), a first-side narrow segment mesh and/or elastic band (22006 and 22014, respectively), and a second-side narrow segment mesh and/or elastic band (22007 and 22015, respectively).

In an example, a first-side (e.g. left side) narrow segment mesh and/or elastic band (e.g. 22006) can have a first level of elasticity and/or flexibility, a second-side (e.g. right side) narrow segment mesh and/or elastic band (e.g. 22007) can have a second level of elasticity and/or flexibility, and the second level can be different than the first level. In an example, such a longitudinally-asymmetric (left-side vs. right-side) difference in narrow segment mesh elasticity and/or flexibility can bias the longitudinal axis of segments into curvature as they are inserted into an aneurysm. In an example, this curvature can cause connected wide segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

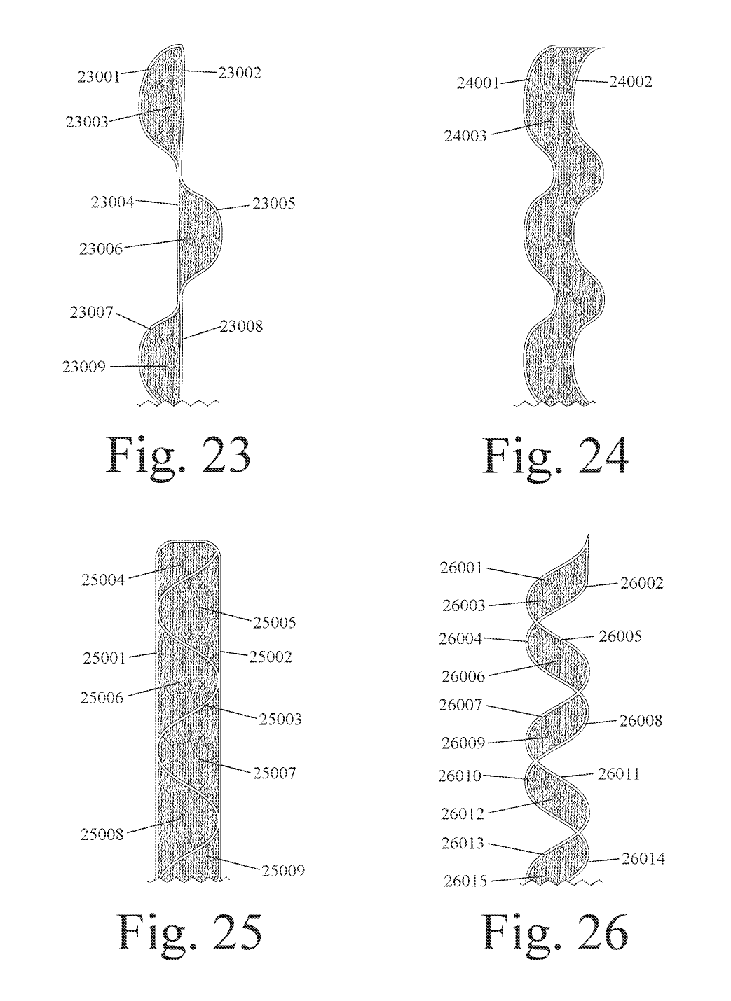

FIG. 23 shows an example of an aneurysm occlusion device with a sequence of single-phase sinusoidal segments (e.g. positive half of sinusoidal cycle and then negative half of sinusoidal cycle) with alternating (left side and then right side) orientations. Three segments are shown here. Each of these three segments has a first-side (e.g. left side) longitudinal wire (23001, 23004, and 23007, respectively), a second-side (e.g. right side) longitudinal wire (23002, 23005, and 23008, respectively), and an inner mesh (23003, 23006, and 23009, respectively) which spans between the first-side and second-side wires. The alternating (left side vs. right side) orientations of segments can bias their longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 24 shows an example of an aneurysm occlusion device with an undulating embolic ribbon comprising a first-side (e.g. left-side) undulating longitudinal wire 24001, a second-side (e.g. right-side) undulating longitudinal wire 24002, and a mesh 24003 between the first-side and second-side longitudinal wires. In an example, undulations can be sinusoidal. In an example, a ribbon's undulations can bias it into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 25 shows an example of an aneurysm occlusion device with an embolic ribbon comprising a first-side (e.g. left-side) longitudinal wire 25001, a second-side (e.g. right-side) longitudinal wire 25002, a central arcuate longitudinal wire 25003 between the first-side and second-side longitudinal wires, and a plurality of mesh sections (25004, 25005, 25006, 25007, 25008, and 25009) between the first-side and second-side longitudinal wires (on different sides of the central arcuate longitudinal wire). In an example, a central arcuate longitudinal wire can be sinusoidal. In an example, a first mesh section (e.g. 25004) on a first side (e.g. the left side) of a central arcuate longitudinal wire can have a first level of elasticity and/or flexibility, a second mesh section (e.g. 25005) on a second side (e.g. the right side) of a central arcuate longitudinal wire can have a second level of elasticity and/or flexibility, and the second level can be different than the first level. In an example, such a longitudinally-asymmetric (left-side vs. right-side) difference in elasticity and/or flexibility can bias a ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 26 shows an example of an aneurysm occlusion device comprising a longitudinal sequence of segments formed by the areas between two intersecting sinusoidal side wires. In this example, the two side wires are sinusoidal with the same amplitude, wavelength, and central longitudinal axis—but are out of phase with each other. In an example, two side wires can be sinusoidal and out of phase by approximately 90 degrees. In an example, two side wires can be sinusoidal and out of phase by a number of degrees within the range of 20 to 160. FIG. 26 shows five segments formed by the intersection of two out-of-phase sinusoidal side wires. Each of the five segments comprises: a first-side (e.g. left side) longitudinal wire portion (26001, 26004, 26007, 26010, and 26013, respectively), a second-side (e.g. right side) longitudinal wire portion (26002, 26005, 26008, 26011, and 26014, respectively), and an inner mesh (26003, 26006, 26009, 26012, and 26015, respectively) between the first-side and second-side wire portions. The alternating orientations of segments formed by the intersecting sinusoidal side wires can bias the longitudinal axis of the sequence into curvature as the segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

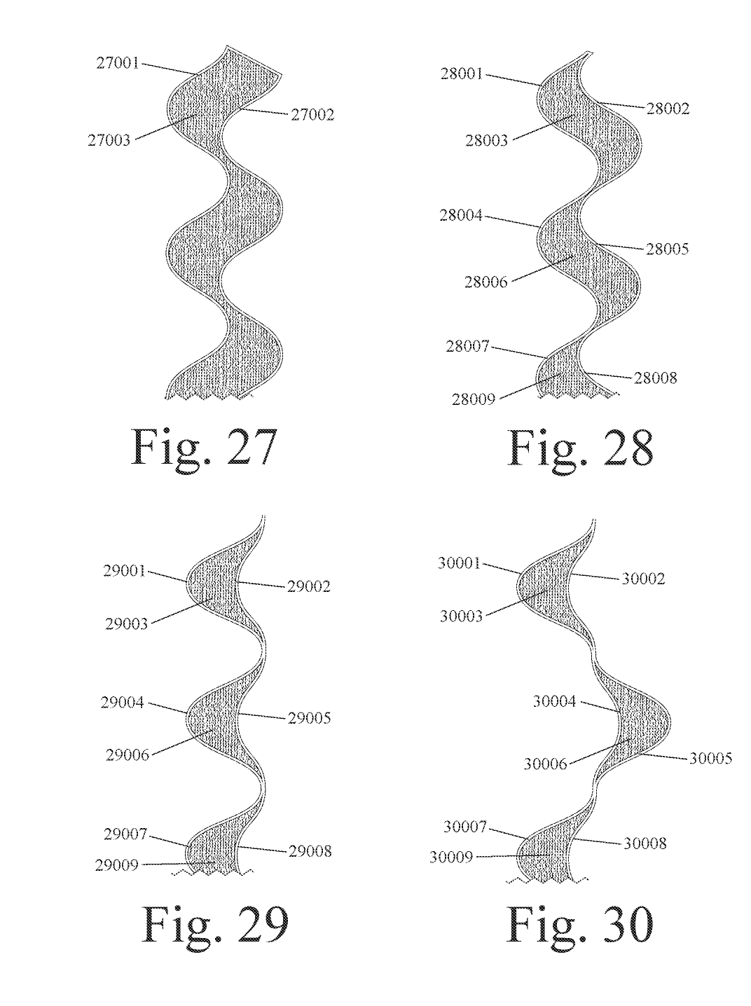

FIG. 27 shows an example of an aneurysm occlusion device comprising an undulating embolic ribbon formed by the area between two non-intersecting sinusoidal side wires which have the same amplitude and wavelength, but have different longitudinal axes and are out of phase with each other. In an example, two non-intersecting sinusoidal side wires can be out of phase by approximately 90 degrees. In an example, two non-intersecting sinusoidal side wires can be out of phase by a number of degrees within the range of 20 to 160. FIG. 27 shows an undulating embolic ribbon comprising a first-side (e.g. left-side) undulating longitudinal wire 27001, a second-side (e.g. right-side) undulating longitudinal wire 27002, and a mesh 27003 between the first-side and second-side longitudinal wires. In an example, a ribbon's undulations can bias it into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 28 shows an example of an aneurysm occlusion device comprising a longitudinal sequence of segments formed by the areas between two tangential sinusoidal side wires. In this example, the two side wires are sinusoidal with the same amplitude and wavelength, but are out of phase with each other by a number of degrees within the range of 20 to 160. FIG. 28 shows three segments formed between two out-of-phase sinusoidal side wires. Each of the three segments comprises: a first-side (e.g. left side) longitudinal wire portion (28001, 28004, and 28007, respectively), a second-side (e.g. right side) longitudinal wire portion (28002, 28005, and 28008, respectively), and an inner mesh (28003, 28006, and 28009, respectively) between the first-side and second-side wire portions. The alternating orientations of segments formed by the sinusoidal side wires can bias the longitudinal axis of the sequence into curvature as the segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 29 shows an example of an aneurysm occlusion device comprising a longitudinal sequence of segments formed by the areas between two tangential sinusoidal side wires with the same wavelength and phase, but different amplitudes. FIG. 29 shows three segments formed the intersection of these two sinusoidal side wires. Each of the three segments comprises: a first-side (e.g. left side) longitudinal wire portion (29001, 29004, and 29007, respectively), a second-side (e.g. right side) longitudinal wire portion (29002, 29005, and 29008, respectively), and an inner mesh (29003, 29006, and 29009, respectively) between the first-side and second-side wire portions. In this example, the first-side longitudinal wire is sinusoidal with a first amplitude, the second-side longitudinal wire is sinusoidal with a second amplitude, and the second amplitude is different than the first amplitude. The longitudinally-asymmetric (right side vs. left side) geometry of these segments can bias their common longitudinal axis into curvature as the segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 30 shows an example of an aneurysm occlusion device with a plurality of longitudinal segments which are geometrically-asymmetric with respect to their longitudinal axes, which each have the same shape, and which differ in orientation. Three segments are shown here. Each of the three segments has a first-side longitudinal wire (30001, 30004, and 30007, respectively), a second-side longitudinal wire (30002, 30005, and 30008, respectively), and an inner mesh (30003, 30006, and 30009, respectively) between the first-side and second-side wires. The geometric-asymmetry of these segments with respect to their longitudinal axes and their differences in orientation can bias their common longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

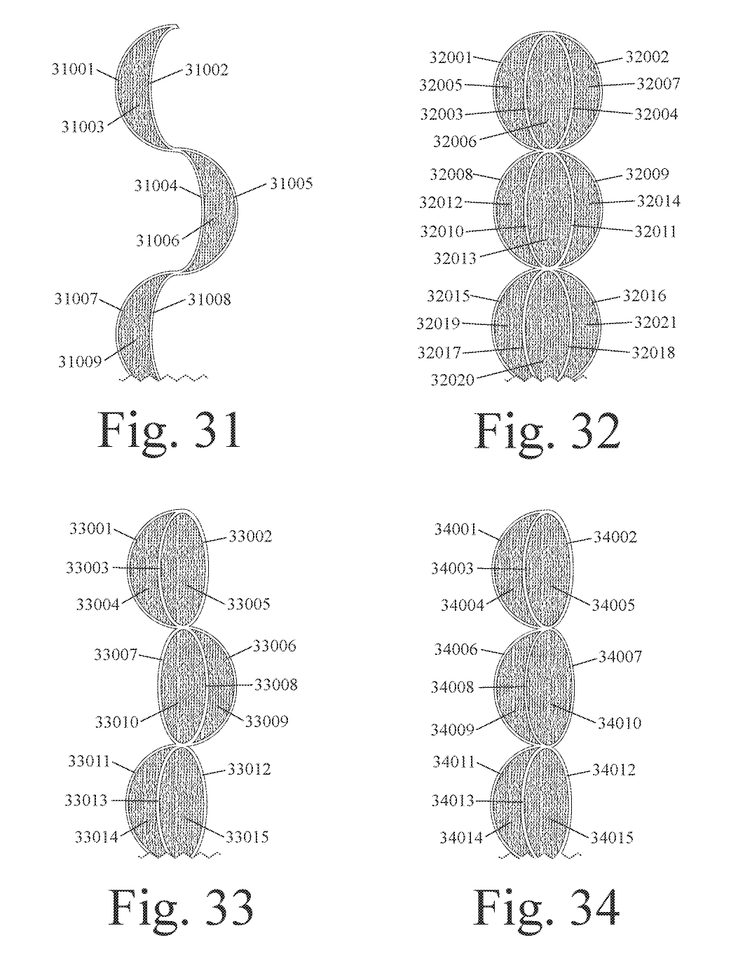

FIG. 31 shows an example of an aneurysm occlusion device with a plurality of connected crescent-shaped longitudinal segments. Three segments are shown here. In this example, crescent-shaped segments have different orientations. In an example, crescent-shaped segments can all have the same orientation. In this example, each of the three segments has a first longitudinal wire (31001, 31004, and 31007, respectively), a second longitudinal wire (31002, 31005, and 31008, respectively), and an inner mesh (31003, 31006, and 31009, respectively) between the first and second wires. The geometric-asymmetry of these segments with respect to their longitudinal axes and their differences in orientation can bias their common longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 32 shows an example of an aneurysm occlusion device with a plurality of connected arcuate segments. Three segments are shown here. In this example, each of the arcuate segments is circular with an inner elliptical loop. In an example, an arcuate segment can be circular, elliptical, or oval. Each of the three segments has a first side exterior wire (32001, 32008, and 32015, respectively), a second side exterior wire (32002, 32009, and 32016, respectively), a first side loop wire (32003, 32010, and 32017, respectively), a second side loop wire (32004, 32011, and 32018, respectively), a first side mesh (32005, 32012, and 32019, respectively) between the first side and second side exterior wires, a second side mesh (32007, 32014, 32021, respectively) between the first side and second side exterior wires, and a central mesh (32006, 32013, and 32020, respectively) between the first side loop wire and the second side loop wire. In an example, a first side mesh (e.g. 32005) on a first side (e.g. the left side) can have a first level of elasticity and/or flexibility, a second mesh section (e.g. 32007) on a second side (e.g. the right side) can have a second level of elasticity and/or flexibility, and the second level can be different than the first level. In an example, such a longitudinally-asymmetric (left-side vs. right-side) difference in elasticity and/or flexibility can bias the longitudinal axis of a plurality of segments into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 33 shows an example of an aneurysm occlusion device with a plurality of connected arcuate segments. Three segments are shown here. Each of the three segments has a first side wire (33001, 33007, and 33011, respectively), a second side wire (33002, 33006, and 33012, respectively), an interior wire (33003, 33008, and 33013, respectively) between the first side and second side wires, a central mesh (33005, 33010, and 33015, respectively), and a side mesh (33004, 33009, and 33014, respectively). In an example, such a longitudinally-asymmetric (left-side vs. right-side) geometry and the different orientations of the segments can bias their longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 34 shows an example of an aneurysm occlusion device with a plurality of connected arcuate segments. Three segments are shown here. Each of the three segments has a first side wire (34001, 34006, and 34011, respectively), a second side wire (34002, 34007, and 34012, respectively), an interior wire (34003, 34008, and 34013, respectively) between the first side and second side wires, a central mesh (34005, 34010, and 34015, respectively), and a side mesh (34004, 34009, and 34014, respectively). In an example, having longitudinally-asymmetric (left-side vs. right-side) segments can bias their common longitudinal axis into curvature as these segments are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

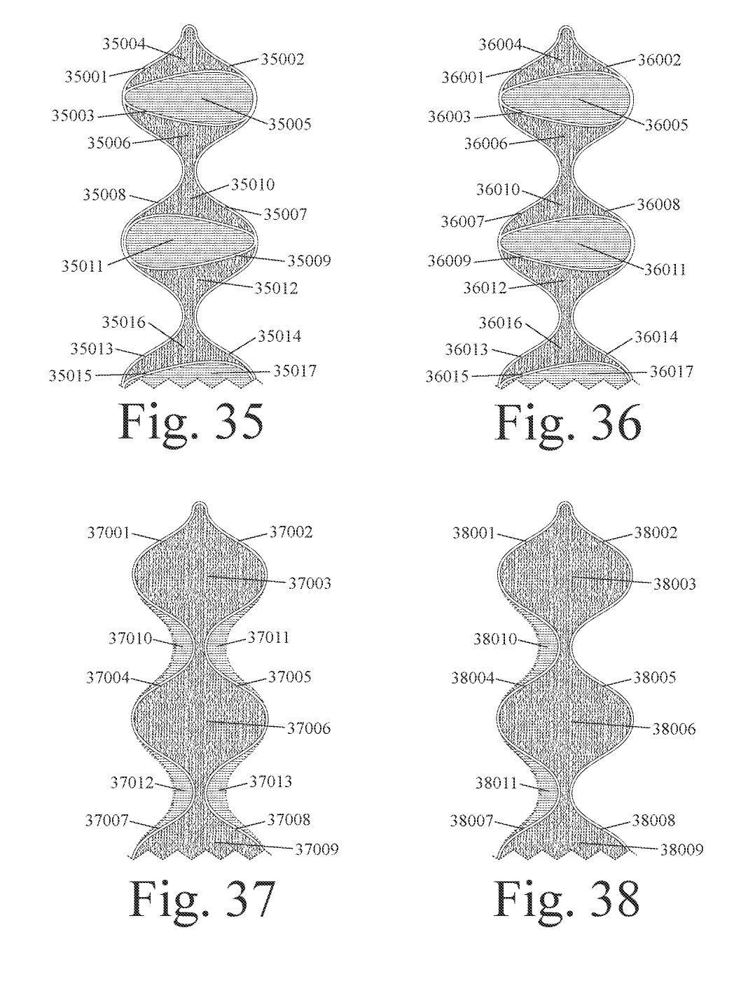

FIG. 35 shows an example of an aneurysm occlusion device with a sequence of (wide and narrow) segments formed by the areas between two non-intersecting sinusoidal (right and left) side wires with the same wavelength and amplitude, but opposite phases (i.e. a 180-degree phase difference). Further, each of the wide segments contains a longitudinally-asymmetric inner loop. Three wide segments are shown. Each of the three wide segments has a first-side longitudinal wire (35001, 35008, and 35013, respectively), a second-side longitudinal wire (35002, 35007, and 35014, respectively), a longitudinally-asymmetric inner wire loop (35003, 35009, and 35015, respectively) between the first-side and second-side wires, one or more outer mesh portions (35004, 35006, 35010, 35012, and 35016) between the first-side and second-side wires, and a inner mesh portion (35005, 35011, and 35017, respectively) within the inner wire loop. In this example, the orientations of the longitudinally-asymmetric loops differ between segments. The longitudinally-asymmetric (left-side vs. right-side) geometry of the inner wire loops and their differing orientations can bias the common longitudinal axis of the segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 36 shows an example of an aneurysm occlusion device with a sequence of (wide and narrow) segments formed by the areas between two non-intersecting sinusoidal (right and left) side wires with the same wavelength and amplitude, but opposite phases (i.e. a 180-degree phase difference). Further, each of the wide segments contains a longitudinally-asymmetric inner loop. Three wide segments are shown. Each of the three wide segments has a first-side longitudinal wire (36001, 36007, and 36013, respectively), a second-side longitudinal wire (36002, 36008, and 36014, respectively), a longitudinally-asymmetric inner wire loop (36003, 36009, and 36015, respectively) between the first-side and second-side wires, one or more outer mesh portions (36004, 36006, 36010, 36012, and 36016) between the first-side and second-side wires, and a inner mesh portion (36005, 36011, and 36017, respectively) within the inner wire loop. In this example, the orientations of the longitudinally-asymmetric loops are the same in all segments. The longitudinally-asymmetric (left-side vs. right-side) geometry of the inner wire loops can bias the common longitudinal axis of the segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 37 shows an example of an aneurysm occlusion device with a sequence of (wide and narrow) segments formed by the areas between two non-intersecting sinusoidal (right and left) side wires with the same wavelength and amplitude, but opposite phases (i.e. a 180-degree phase difference). This example also includes longitudinally-symmetric (e.g. left side and right side) elastic connectors between pairs of wide segments. Three wide segments are shown. Each of the three wide segments has a first-side longitudinal wire (37001, 37004, and 37007, respectively), a second-side longitudinal wire (37002, 37005, and 37008, respectively), and a mesh (37003, 37006, and 37009, respectively) between the first-side and second-side longitudinal wires. There are also first-side (e.g. left-side) elastic connectors (37010 and 37012) and second-side (e.g. right-side) elastic connectors (37011 and 37013) between pairs of wide segments. In an example, a first-side elastic connector (e.g. 37010) can have a first level of elasticity and/or flexibility, a second-side elastic connector (e.g. 37011) can have a second level of elasticity and/or flexibility, and the second level can be different than the first level. In an example, such a longitudinally-asymmetric (left-side vs. right-side) difference in elasticity and/or flexibility can bias the common longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 38 shows an example of an aneurysm occlusion device with a sequence of (wide and narrow) segments formed by the areas between two non-intersecting sinusoidal (right and left) side wires with the same wavelength and amplitude, but opposite phases (i.e. a 180-degree phase difference). This example also includes longitudinally-asymmetric (e.g. left side only) elastic connectors between pairs of wide segments. Three wide segments are shown. Each of the three wide segments has a first-side longitudinal wire (38001, 38004, and 38007, respectively), a second-side longitudinal wire (38002, 38005, and 38008, respectively), and a mesh (38003, 38006, and 38009, respectively) between the first-side and second-side longitudinal wires. There are also first-side only (e.g. left-side only) elastic connectors 38010 and 38011 between pairs of wide segments. In an example, longitudinally-asymmetric (left-side only) elastic connectors can bias the common longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

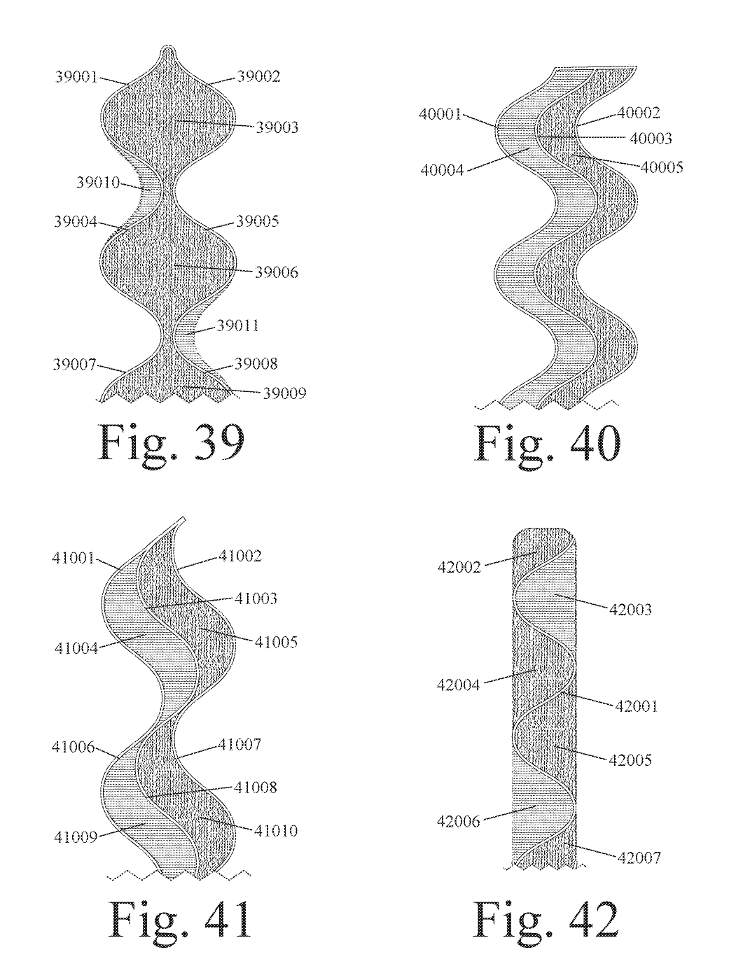

FIG. 39 shows an example of an aneurysm occlusion device with a sequence of (wide and narrow) segments formed by the areas between two non-intersecting sinusoidal (right and left) side wires with the same wavelength and amplitude, but opposite phases (i.e. a 180-degree phase difference). This example also includes longitudinally-asymmetric (e.g. alternating left-side and right-side) elastic connectors between pairs of wide segments. Three wide segments are shown. Each of the three wide segments has a first-side longitudinal wire (39001, 39004, and 39007, respectively), a second-side longitudinal wire (39002, 39005, and 39008, respectively), and a mesh (39003, 39006, and 39009, respectively) between the first-side and second-side longitudinal wires. There is one left-side elastic connector 39010 between one pair of wide segments and one right-side elastic connector 39011 between another pair of wide segments. In an example, such longitudinally-asymmetric elastic connectors can bias the common longitudinal axis of segments into curvature as they are inserted into an aneurysm. In an example, this curvature can cause connected segments to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 40 shows an example of an aneurysm occlusion device with an undulating embolic ribbon comprising a first-side (e.g. left-side) undulating wire 40001, a second-side (e.g. right-side) undulating wire 40002, a central undulating wire 40003 between the first-side and second-side undulating wires, a first-side mesh and/or elastic band 40004 between the first-side undulating wire and the central undulating wire, and a second-side mesh and/or elastic band 40005 between the second-side undulating wire and the central undulating wire. In this example, the first-side and second-side undulating wires are non-intersecting and sinusoidal, with the same wavelength, amplitude, and phase. They are also parallel to each other. In an example, a ribbon's undulations can bias it into curvature as it is inserted into an aneurysm. In an example, a first-side mesh and/or elastic band can have a first level of elasticity and/or flexibility, a second-side mesh and/or elastic band can have a second level of elasticity and/or flexibility, and the second level can be different than the first level. In an example, a longitudinally-asymmetric (left-side vs. right-side) difference in elasticity and/or flexibility can bias a ribbon into curvature as it is inserted into an aneurysm. In an example, such curvature can cause a ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 41 shows an example of an aneurysm occlusion device with an undulating embolic ribbon comprising “flame-shaped” wide and narrow segments. Two wide segments are shown here. Each of the two wide segments includes a first-side (e.g. left-side) undulating wire (41001 and 41006, respectively), a second-side (e.g. right-side) undulating wire (41002 and 41007, respectively), a central undulating wire (41003 and 41008, respectively) between the first-side and second-side undulating wires, a first-side mesh and/or elastic band (41004 and 41009, respectively) between the first-side undulating wire and the central undulating wire, and a second-side mesh and/or elastic band (41005 and 41010, respectively) between the second-side undulating wire and the central undulating wire. In this example, the undulating wires are non-intersectional and sinusoidal, with a common wavelength and amplitude, but they are out-of-phase with each other. In an example, they are out-of-phase by a number of degrees ranging from 20 to 160 degrees. In an example, a ribbon's charismatic undulations can bias it into curvature as it is inserted into an aneurysm. In an example, a first-side mesh and/or elastic band can have a first level of elasticity and/or flexibility, a second-side mesh and/or elastic band can have a second level of elasticity and/or flexibility, and the second level can be different than the first level. In an example, a longitudinally-asymmetric (left-side vs. right-side) difference in elasticity and/or flexibility can bias a ribbon into curvature as it is inserted into an aneurysm. In an example, such curvature can cause a ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 42 shows an example of an aneurysm occlusion device with an embolic ribbon comprising a central undulating wire 42001, a plurality of first-side (e.g. left-side) mesh and/or elastic sections (including 42002, 42004, and 42006) filling the (convex or concave) areas between curves on the first side of the central undulating wire, and a plurality of second-side (e.g. right-side) mesh and/or elastic sections (including 42003, 42005, and 42007) filling the (concave or convex) areas between curves on the second side of the central undulating wire. In an example, a central undulating wire can be sinusoidal. In an example, a first-side mesh and/or elastic section (e.g. 42002) can have a first level of elasticity and/or flexibility, a second-side mesh and/or elastic section (e.g. 42003) can have a second level of elasticity and/or flexibility, and the second level can be different than the first level. In an example, one first-side mesh and/or elastic section (e.g. 42002) can have a first level of elasticity and/or flexibility, a different first-side mesh and/or elastic section (e.g. 42006) can have a second level of elasticity and/or flexibility, and the second level can be different than the first level. In an example, such differences in elasticity and/or flexibility can bias a ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

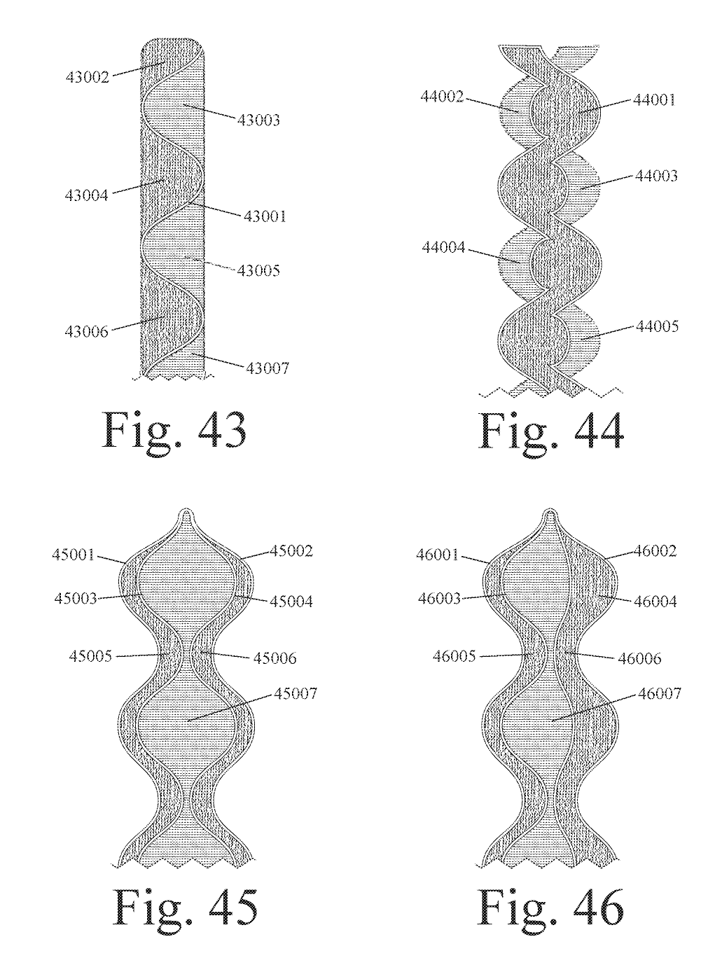

FIG. 43 shows an example of an aneurysm occlusion device with an embolic ribbon comprising a central undulating wire 43001, a plurality of first-side (e.g. left-side) mesh and/or elastic sections (including 43002, 43004, and 43006) filling the (convex or concave) areas between curves on the first side of the central undulating wire, and a plurality of second-side (e.g. right-side) mesh and/or elastic sections (including 43003, 43005, and 43007) filling the (concave or convex) areas between curves on the second side of the central undulating wire. In an example, a central undulating wire can be sinusoidal. In an example, first-side meshes and/or elastic sections can have a first level of elasticity and/or flexibility, second-side meshes and/or elastic sections can have a second level of elasticity and/or flexibility, and the second level can be different than the first level. In an example, such a longitudinally-asymmetric (left-side vs. right-side) difference in elasticity and/or flexibility can bias a ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 44 is a weird design that will probably never see the light of day, but Al might like it. Did you know that Al was valedictorian of his class at Lynwood High School? He does amazing parodies. Ah well, now back to aneurysm occlusion devices. FIG. 44 is an aneurysm occlusion device comprising: a sinusoidal ribbon 44001 with intra-curve bulges (looking like a series of apostrophes or commas with alternating orientations); and a plurality of inter-curve elastic meshes or bands (44002, 44003, 44004, and 44005). In an example, longitudinally-asymmetric (left-side vs. right-side) differences in elasticity and/or flexibility can bias a ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 45 shows an example of an aneurysm occlusion device with a sequence of wide and narrow segments formed by two sinusoidally-undulating ribbons with the same central longitudinal axis. In this example, each the ribbon is formed by the area between two sinusoidal side wires, wherein the two sinusoidal side wires have the same wavelength and amplitude, but opposite phases (i.e. a 180-degree phase difference). The first (outer) ribbon has a first width and the second (inner) ribbon has a second width. The first width is greater than the second width. FIG. 45 shows a first-side (e.g. left-side) sinusoidal wire 45001 of the first (outer) ribbon, a second-side (e.g. right-side) sinusoidal wire 45002 of the first (outer) ribbon, a first-side sinusoidal wire 45003 of the second (inner) ribbon, a second-side sinusoidal wire 45004 of the second (inner) ribbon, a first-side mesh 45005 between first and second ribbons, a second-side mesh 45006 between the first and second ribbons, and an inner mesh 45007 within the second ribbon. In an example, a first-side mesh can have a first level of elasticity and/or flexibility, a second-side mesh can have a second level of elasticity and/or flexibility, and the second level can be different than the first level. In an example, such a longitudinally-asymmetric (left-side vs. right-side) difference in elasticity and/or flexibility can bias the longitudinal axis of the ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

FIG. 46 shows an example of an aneurysm occlusion device like the one in FIG. 45 except that the second (inner) ribbon is longitudinally asymmetric. FIG. 46 shows a first-side (e.g. left-side) sinusoidal wire 46001 of the first (outer) ribbon, a second-side (e.g. right-side) sinusoidal wire 46002 of the first (outer) ribbon, a first-side sinusoidal wire 46003 of the second (inner) ribbon, a second-side sinusoidal wire 46004 of the second (inner) ribbon, a first-side mesh 46005 between first and second ribbons, a second-side mesh 46006 between the first and second ribbons, and an inner mesh 46007 within the second ribbon. In an example, the longitudinal asymmetry of the second (inner) ribbon can bias the longitudinal axis of the ribbon into curvature as it is inserted into an aneurysm. In an example, this curvature can cause the ribbon to accumulate into a spherical, elliptical, or other arcuate mass which occludes the aneurysm. Example variations and descriptions discussed elsewhere in this disclosure or in priority-linked disclosures can be applied to this example, but are not repeated here in order to reduce redundancy.

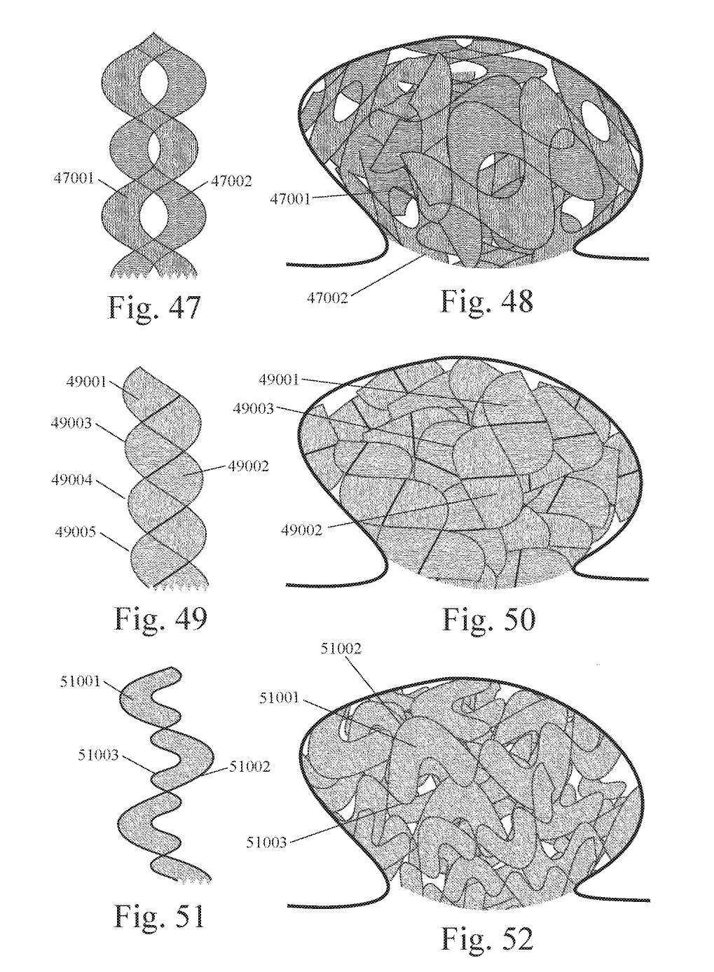

FIGS. 47 and 48 show two sequential views of an aneurysm occlusion device comprising an undulating embolic ribbon which is configured to be inserted into an aneurysm sac. This undulating embolic ribbon is configured to accumulate into an arcuate three-dimensional occlusive mass which fills an aneurysm sac. This undulating embolic ribbon further comprises a first undulating strip 47001 and a second undulating strip 47002. In this example, the second undulating strip is a reflected version of the first undulating strip, having been reflected across a longitudinal axis of the undulating embolic ribbon.

FIG. 47 shows this device at a first point in time, before it has been inserted into an aneurysm sac. FIG. 48 shows this device at a second point in time, after it has been inserted into an aneurysm sac and accumulated in an overlapping manner into an arcuate three-dimensional occlusive mass which fills an aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal.

Expressing this embodiment with different words, FIGS. 47 and 48 show an aneurysm occlusion device comprising: an undulating embolic ribbon which is configured to be inserted into an aneurysm sac so as to accumulate into an arcuate three-dimensional occlusive mass within the aneurysm sac, wherein the undulating embolic ribbon further comprises a first undulating strip and a second undulating strip, and wherein the first and second undulating strips are symmetric relative to each other across a longitudinal axis of the undulating embolic ribbon. In an example, an undulating strip can be sinusoidal. In an example, this aneurysm occlusion device can comprise: an undulating embolic ribbon which is configured to be inserted into an aneurysm sac so as to accumulate into an arcuate three-dimensional occlusive mass within the aneurysm sac, wherein the undulating embolic ribbon further comprises a first sinusoidal strip and a second sinusoidal strip.

In an example, a sinusoidal strip can have a constant wavelength along its entire length. In an example, the wavelength of a distal portion of a sinusoidal strip can be greater than the wavelength of a proximal portion of a sinusoidal strip, or vice versa. In an example, a sinusoidal strip can have a constant width along its entire length. In an example, the width of a distal portion of a sinusoidal strip can be greater than the width of a proximal portion of a sinusoidal strip, or vice versa. In an example, a sinusoidal strip can have a constant thickness along its entire length. In an example, the thickness of a distal portion of a sinusoidal strip can be greater than the thickness of a proximal portion of a sinusoidal strip, or vice versa. In an example, a sinusoidal strip can have a constant elasticity and/or flexibility level along its entire length. In an example, the elasticity and/or flexibility level of a distal portion of a sinusoidal strip can be greater than the elasticity and/or flexibility level of a proximal portion of a sinusoidal strip, or vice versa.

In an example, an undulating strip can be made from a metal, a polymer, or both. In an example, an undulating strip can comprise a wire mesh, net, or lattice. In an example, an undulating strip can further comprise two or more undulating wires with a mesh, net, or lattice between them. In an example, an undulating strip can further comprise two or more undulating wires with fabric between them. In an example, there can be gaps between first and second undulating strips. In an example, these gaps can vary sequentially in shape. In an example, there may be no gaps between the first and second undulating strips. In an example, first and second undulating strips can overlap. In an example, the first and second undulating strips can be attached to each other.

In an example, an undulating embolic ribbon can have cross-sectional asymmetry. In an example, this cross-sectional asymmetry can be due to cross-sectional differences in elasticity, flexibility, shape, length, and/or width. In an example, a first undulating strip can have a first elasticity level and a second undulating strip can have a second elasticity level, wherein the second elasticity level is greater than the first elasticity level. In an example, a first undulating strip can have a first flexibility level and a second undulating strip can have a second flexibility level, wherein the second flexibility level is greater than the first flexibility level. In an example, a first undulating strip can have a first width and a second undulating strip can have a second width, wherein the second width is greater than the first width.

In an example, the cross-sectional asymmetry of an undulating embolic ribbon can bias the embolic ribbon to bend to the right or to the left as it exits a catheter. Such bending can cause the undulating embolic ribbon to form an arcuate three-dimensional occlusive mass as it accumulates within an aneurysm sac. In an example, the cross-sectional asymmetry of an undulating embolic ribbon can bias the embolic ribbon to bend sequentially back and forth, oscillating to the right and to the left. Such oscillating bending can cause an undulating embolic ribbon to form an arcuate three-dimensional mass as it accumulates within an aneurysm sac. In an example, an arcuate three-dimensional mass occlusive formed with an aneurysm sac can be generally spherical, ellipsoidal, or ovaloidal in shape. In an example, an arcuate three-dimensional occlusive mass can also have bulges and/or bumps so as conform to the walls of an irregularly-shaped (e.g. non-spherical) aneurysm sac.

In an example, the cross-sectional symmetry of an undulating embolic ribbon can be adjusted by a user in real-time (as an embolic ribbon is being inserted into an aneurysm sac) so as to guide and/or steer the bending movement of the ribbon as it is deployed. This enables a user to guide the formation of an arcuate three-dimensional occlusive mass which confirms to the walls of an irregularly-shaped aneurysm sac. In an example, a user can adjust the cross-sectional asymmetry of an undulating embolic ribbon by adjusting its elasticity, flexibility, shape, length, and/or width as it is deployed within an aneurysm sac. In an example, application of electromagnetic energy to a first undulating strip and/or to a second undulating strip can change the cross-sectional asymmetry of the undulating embolic ribbon, thereby biasing the embolic ribbon to bend to the right or to the left. In an example, pulling or pushing a wire connected to the first undulating strip or connected to the second undulating strip can bias the undulating embolic ribbon to bend to the right or to the left.

In an example, this device can further comprise an electromagnetic energy source which enables a user to selectively apply electromagnetic energy to a portion of an embolic ribbon as the ribbon is deployed within an aneurysm sac. In an example, selective application of electromagnetic energy to a portion of an embolic ribbon can change the elasticity, flexibility, and/or shape of that portion. In an example, selective application of electromagnetic energy to a first side of an embolic ribbon can cause the ribbon to bend in a first direction and selective application of electromagnetic energy to a second side of the embolic ribbon can cause the ribbon to bend in a second direction. In an example, this enables a user to guide and/or steer the embolic ribbon during deployment so as to create an arcuate three-dimensional occlusive mass which optimally fills an aneurysm sac.

In an example, an undulating embolic ribbon can accumulate within an aneurysm sac so as to form an arcuate three-dimensional occlusive mass, progressively filling it from the outside of the mass to the inside of the mass. In an example, a catheter dispensing an undulating embolic ribbon can be positioned in the center of an aneurysm sac so as to form an arcuate three-dimensional occlusive mass starting from the outside of the mass and then progressively filling the inside.

In an example, an undulating embolic ribbon can accumulate within an aneurysm sac so as to form an arcuate three-dimensional occlusive mass, progressing from the inside of the mass to the outside of the mass, like wrapping a ball of yarn. In an example, a catheter dispensing an undulating embolic ribbon can be positioned near the wall of an aneurysm sac so as to form an arcuate three-dimensional occlusive mass starting from the inside of the mass and then progressively covering the outside, like wrapping a ball of yarn. Relevant design variations discussed elsewhere in this disclosure or in priority-linked disclosures can also be applied to the example shown here.

FIGS. 49 and 50 show two sequential views of an aneurysm occlusion device comprising an undulating embolic ribbon which is configured to be inserted into an aneurysm sac. This undulating embolic ribbon is configured to accumulate into an arcuate three-dimensional occlusive mass which fills an aneurysm sac. In this example, the undulating embolic ribbon further comprises: a first set of pie-slice portions (shaped like slices of pie), including 49001, along the left side of the ribbon; and a second set of pie-slice portions, including 49002, along the right side of the ribbon.

FIG. 49 shows this device at a first point in time, before it has been inserted into an aneurysm sac. FIG. 50 shows this device at a second point in time, after it has been inserted into an aneurysm sac and accumulated in an overlapping manner into an arcuate three-dimensional occlusive mass which fills the aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal.

In an example, pie-slice portions in a first set or in a second set can be contiguous to each other. In an example, pie-slice portions in first and second sets can interdigitate. In an example, the rounded edges of pie-slice portions can point away from the central longitudinal axis of an embolic ribbon. In an example, pie-slice portions in a second set can be made from a different material than pie-slice portions in a first set. In an example, pie-slice portions in a second set can have a different elasticity, thickness, width, and/or size than pie-slice portions in a first set. In an example, differences in material characteristics between pie-slice portions in first and second sets can bias an embolic ribbon to bend to one side (or the other) as it exits a catheter within an aneurysm sac in order to form an arcuate three-dimensional occlusive mass which fills the aneurysm sac.

This example can also be described as comprising three undulating wires, 49003, 49004, and 49005. In this example, these undulating wires are sinusoidal. In this example, there is occluding mesh or fabric between the wires. In an example, multiple sinusoidal wires in an embolic ribbon can share the same central longitudinal axis. In an example, multiple sinusoidal wires can have the same wavelength, but have different phases. In an example, the phase of a second sinusoidal wire can differ from the phase of a first sinusoidal wire by 120 degrees and the phase of a third sinusoidal wire can differ from the phase of a first sinusoidal wire by 240 degrees. In an example, the phase of a second sinusoidal wire can differ from the phase of a first sinusoidal wire by 60 degrees and the phase of a third sinusoidal wire can differ from the phase of a first sinusoidal wire by 120 degrees.

In an example, an embolic ribbon can have the same width along its entire length. In an example, a proximal portion of an embolic ribbon can be wider than its distal portion, or vice versa. In an example, an embolic ribbon can have the same thickness along its entire length. In an example, a proximal portion of an embolic ribbon can be thicker than its distal portion, or vice versa. In an example, an embolic ribbon can have the same elasticity level along its entire length. In an example, a proximal portion of an embolic ribbon can be more elastic than its distal portion, or vice versa.

In an example, there can be variation in cross-sectional differences in material characteristics in an embolic ribbon along its longitudinal axis. In an example, there can be variation in material characteristics between first set and second set portions along the longitudinal axis of an embolic ribbon. In an example, portions in a first set can be more flexible, thicker, or wider along a first segment of the longitudinal axis of a ribbon and portions in a second set can be more flexible, thicker, or wider along a second segment of the longitudinal axis of the ribbon.

In an example, variation in cross-sectional differences can cause an embolic ribbon to bend in different directions along different sections of its longitudinal axis as it exits a catheter within an aneurysm sac. In an example, variation in cross-sectional differences can cause an embolic ribbon to oscillate between bending to the left and bending to the right as it exits a catheter within an aneurysm sac. In an example, variation in differences between first and second sets can cause an embolic ribbon to oscillate and/or alternate between bending to the left and bending to the right as it exits a catheter within an aneurysm sac.

In an example, this device can further comprise an electromagnetic energy source which enables a user to selectively apply electromagnetic energy to a first set or to a second set of pie-shape portions as an embolic ribbon is deployed within an aneurysm sac. In an example, selective application of electromagnetic energy can change the elasticity, flexibility, and/or shape of a first set or a second set. In an example, selective application of electromagnetic energy to a first side of an embolic ribbon can cause the ribbon to bend in a first direction and selective application of electromagnetic energy to a second side of the embolic ribbon can cause the ribbon to bend in a second direction. In an example, this enables a user to guide and/or steer the embolic ribbon during deployment so as to create an arcuate three-dimensional occlusive mass which optimally fills an aneurysm sac. Relevant design variations discussed elsewhere in this disclosure or in priority-linked disclosures can also be applied to the example shown here.

FIGS. 51 and 52 show two sequential views of an aneurysm occlusion device comprising an undulating embolic ribbon which is configured to be inserted into an aneurysm sac. This undulating embolic ribbon 51001 is configured to accumulate into an arcuate three-dimensional occlusive mass which fills the aneurysm sac. In this example, the undulating embolic ribbon further comprises: a first sinusoidal wire 51002 with a first wavelength; a second sinusoidal wire 51003 with a second wavelength, wherein the second wavelength is half of the first wavelength; and an occlusive mesh or fabric between the first sinusoidal wire and the second sinusoidal wire. In this example, the first and second sinusoidal wires share a common central longitudinal axis.

FIG. 51 shows this device at a first point in time, before it has been inserted into an aneurysm sac. FIG. 52 shows this device at a second point in time, after it has been inserted into an aneurysm sac and accumulated in an overlapping manner into an arcuate three-dimensional occlusive mass which fills the aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal. Relevant design variations discussed elsewhere in this disclosure or in priority-linked disclosures can also be applied to the example shown here.

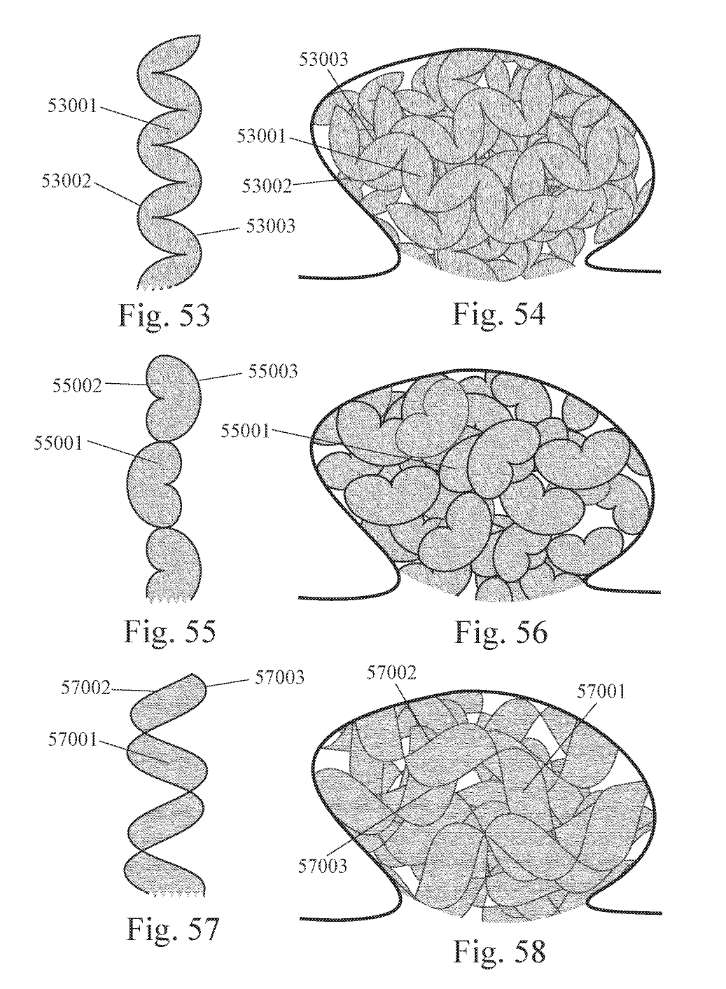

FIGS. 53 and 54 show two sequential views of an aneurysm occlusion device comprising an undulating embolic ribbon 53001 which is configured to be inserted into an aneurysm sac. This undulating embolic ribbon is configured to accumulate into an arcuate three-dimensional occlusive mass which fills the aneurysm sac. In this example, an undulating embolic ribbon further comprises: a first longitudinal series of (pair-wise) connected arcs 53002 comprising a first-side (e.g. left side) perimeter of the ribbon; a second longitudinal series of (pair-wise) connected arcs 53003 comprising a second-side (e.g. right side) perimeter of the ribbon; and an occlusive mesh or fabric between the first longitudinal series and the second longitudinal series.

FIG. 53 shows this device at a first point in time, before it has been inserted into an aneurysm sac. FIG. 54 shows this device at a second point in time, after it has been inserted into an aneurysm sac and accumulated in an overlapping manner into an arcuate three-dimensional occlusive mass which fills the aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal.

In an example, an arc can be a semi-circle or other segment of a circle. In an example, an arc can have a centenary shape. In an example, an arc can be a (180-degree) segment of a sinusoidal curve. In an example, a longitudinal series of connected arcs can comprise a wire. In an example, the left and right sides of an embolic ribbon can be comprises of two longitudinal wires, each of which is a longitudinal series of connected arcs. In an example, an embolic ribbon can further comprise a central wire between the two longitudinal wires on the left and right rides of the ribbon. In an example, a central wire can have a sinusoidal or other undulating shape.

In an example, connected arcs within a longitudinal series can be oriented in the same direction. In an example, connected arcs in a longitudinal series can have convexities which face in the same direction. In an example, connected arcs in a first longitudinal series can be convex in a first direction, connected arcs in a second longitudinal series can be convex in a second direction, and the first and second directions can be opposites of each other. In an example, a second longitudinal series of connected arcs can be vertically reflected and phase shifted relative to a first longitudinal series of connected arcs. In an example, this phase shift can be 90 degrees. In an example, this phase shift can be 180 degrees.

In an example, the closest distances between first and second longitudinal series of connected arcs can occur where arcs within a series connect to each other. In this example, the closest distance is greater than zero. In another example, the closest distance can be zero, meaning that the first and second series contact each other. In an example, this device can form and/or comprise a longitudinal series of mesh or fabric segments, wherein the shape of each mesh or fabric segment in the series is selected from the group consisting of: convex lens; football; leaf; flower petal; stylized eye outline; tear drop; oval; and ellipse.

In an example, adjacent mesh or fabric segments in a longitudinal series of mesh or fabric segments can have different orientations. In an example, a series of mesh or fabric segments can have oscillating and/or alternating (e.g. right vs. left) orientations. In an example, a series of mesh or fabric segments can comprise a longitudinal zigzag pattern. In an example, a series of mesh or fabric segments can have longitudinal axes which zigzag relative to each other, forming 90-degree angles where their axes (or extensions thereof in space) intersect. In an example, a series of mesh or fabric segments can have longitudinal axes, wherein their axes (or extensions thereof in space) intersect at angles within the range of 60 to 120 degrees.

In an example, an embolic ribbon can have the same width along its entire length. In an example, a proximal portion of an embolic ribbon can be wider than its distal portion, or vice versa. In an example, an embolic ribbon can have the same thickness along its entire length. In an example, a proximal portion of an embolic ribbon can be thicker than its distal portion, or vice versa. In an example, an embolic ribbon can have the same elasticity level along its entire length. In an example, a proximal portion of an embolic ribbon can be more elastic than its distal portion, or vice versa.

In an example, an aneurysm occlusion device can further comprise an electromagnetic energy source which enables a user to selectively apply electromagnetic energy to a left-side portion or to a right-side portion of an embolic ribbon as it is deployed within an aneurysm sac. In an example, selective application of electromagnetic energy can change the elasticity, flexibility, and/or shape of a left-side portion or a right-side portion of the ribbon. In an example, selective application of electromagnetic energy to a first side of an embolic ribbon can cause the ribbon to bend in a first direction and selective application of electromagnetic energy to a second side of the embolic ribbon can cause the ribbon to bend in a second direction. In an example, this can enable a user to guide and/or steer an embolic ribbon during deployment so as to create an arcuate three-dimensional occlusive mass which optimally fills an aneurysm sac.

In an example, an embolic ribbon can accumulate within an aneurysm sac so as to form an arcuate three-dimensional occlusive mass, progressively filling it from the outside of the mass to the inside of the mass. In an example, a catheter dispensing an embolic ribbon can be positioned in the center of an aneurysm sac so as to form an arcuate three-dimensional occlusive mass starting from the outside of the mass and then progressively filling the inside.

In an example, an embolic ribbon can accumulate within an aneurysm sac so as to form an arcuate three-dimensional occlusive mass, progressing from the inside of the mass to the outside of the mass, like wrapping a ball of yarn. In an example, a catheter dispensing an embolic ribbon can be positioned near the wall of an aneurysm sac so as to form an arcuate three-dimensional occlusive mass starting from the inside of the mass and then progressively covering the outside, like wrapping a ball of yarn. Relevant design variations discussed elsewhere in this disclosure or in priority-linked disclosures can also be applied to the example shown here.

FIGS. 55 and 56 show two sequential views of an aneurysm occlusion device comprising an undulating embolic ribbon which is configured to be inserted into an aneurysm sac. This undulating embolic ribbon is configured to accumulate into an arcuate three-dimensional occlusive mass which fills the aneurysm sac. In an example, this undulating embolic ribbon can be described as comprising a connected longitudinal series of cardioid or kidney shaped mesh or fabric segments, including 55001. In this example, adjacent cardioid or kidney shaped segments face in opposite directions. In an example, this undulating embolic ribbon can be described as comprising: a first wire 55002 comprising a longitudinal series of (pair-wise) connected arcs; a second wire 55003 which is sinusoidal; and an occlusive mesh or fabric between the first wire and the second wire.

FIG. 55 shows this device at a first point in time, before it has been inserted into an aneurysm sac. FIG. 56 shows this device at a second point in time, after it has been inserted into an aneurysm sac and accumulated in an overlapping manner into an arcuate three-dimensional occlusive mass which fills an aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal. Relevant design variations discussed elsewhere in this disclosure or in priority-linked disclosures can also be applied to the example shown here.

FIGS. 57 and 58 show two sequential views of an aneurysm occlusion device comprising an undulating embolic ribbon 57001 which is configured to be inserted into an aneurysm sac. This undulating embolic ribbon is configured to accumulate into an arcuate three-dimensional occlusive mass which fills the aneurysm sac. In this example, an undulating embolic ribbon further comprises: a first longitudinal sinusoidal wire 57002; a second longitudinal sinusoidal wire 57003, wherein the second longitudinal sinusoidal wire is phase-shifted relative to the first sinusoidal wire; and an occlusive mesh or fabric between the first longitudinal sinusoidal wire and the second longitudinal sinusoidal wire.

In an example, a phase shift between a first longitudinal sinusoidal wire and a second longitudinal sinusoidal wire can be 90 degrees. In an example, a phase shift between a first longitudinal sinusoidal wire and a second longitudinal sinusoidal wire can be between 20 and 160 degrees. In this example, the first longitudinal sinusoidal wire and the second longitudinal sinusoidal wire have the same wavelength. In this example, the first longitudinal sinusoidal wire and the second longitudinal sinusoidal wire have the same amplitude. In this example, the first longitudinal sinusoidal wire and the second longitudinal sinusoidal wire share the same central longitudinal axis. In another example, a first longitudinal sinusoidal wire and a second longitudinal sinusoidal wire can have different wavelengths. In another example, a first longitudinal sinusoidal wire and a second longitudinal sinusoidal wire can have different amplitudes. In another example, a first longitudinal sinusoidal wire and a second longitudinal sinusoidal wire can have different central longitudinal axes.

FIG. 57 shows this device at a first point in time, before it has been inserted into an aneurysm sac. FIG. 58 shows this device at a second point in time, after it has been inserted into an aneurysm sac and accumulated in an overlapping manner into an arcuate three-dimensional occlusive mass which fills the aneurysm sac. In an example, an arcuate three-dimensional occlusive mass formed within an aneurysm sac can have a shape selected from the group consisting of: sphere; ellipsoid; ovaloid; pumpkin; apple; pear; and torus. In an example, an arcuate three-dimensional occlusive mass can also have bulges and bumps which enable it to conform to the walls of an irregularly-shaped aneurysm sac. This is especially important for aneurysms which are not perfectly spherical, ellipsoidal, or ovaloidal.

In an example, an embolic ribbon can have the same width along its entire length. In an example, a proximal portion of an embolic ribbon can be wider than its distal portion, or vice versa. In an example, an embolic ribbon can have the same thickness along its entire length. In an example, a proximal portion of an embolic ribbon can be thicker than its distal portion, or vice versa. In an example, an embolic ribbon can have the same elasticity level along its entire length. In an example, a proximal portion of an embolic ribbon can be more elastic than its distal portion, or vice versa.

In an example, an embolic ribbon can accumulate within an aneurysm sac so as to form an arcuate three-dimensional occlusive mass, progressively filling it from the outside of the mass to the inside of the mass. In an example, a catheter dispensing an embolic ribbon can be positioned in the center of an aneurysm sac so as to form an arcuate three-dimensional occlusive mass starting from the outside of the mass and then progressively filling the inside.

In an example, an embolic ribbon can accumulate within an aneurysm sac so as to form an arcuate three-dimensional occlusive mass, progressing from the inside of the mass to the outside of the mass, like wrapping a ball of yarn. In an example, a catheter dispensing an embolic ribbon can be positioned near the wall of an aneurysm sac so as to form an arcuate three-dimensional occlusive mass starting from the inside of the mass and then progressively covering the outside, like wrapping a ball of yarn. Relevant design variations discussed elsewhere in this disclosure or in priority-linked disclosures can also be applied to the example shown here.

In an example, a device for occluding an aneurysm can comprise: a first longitudinal wire which is inserted into an aneurysm sac; a second longitudinal wire which is inserted into the aneurysm sac, wherein the first and second longitudinal wires intersect, overlap, or connect at least three times along their longitudinal axes, forming at least two wire loops between the first and second longitudinal wires; and a mesh material which spans the at least two loops. In an example, a device for occluding an aneurysm can comprise: a first longitudinal wire which is inserted into an aneurysm sac; a second longitudinal wire which is inserted into the aneurysm sac, wherein the first and second longitudinal wires converge and diverge at least three times along their longitudinal axes, forming at least two arcuate areas between the first and second longitudinal wires; and a mesh material which spans the at least two arcuate areas.

In an example, the first and second longitudinal wires can be undulating or sinusoidal. In an example, the first and second longitudinal wires can be sinusoidal and out-of-phase with each other. In an example, the device can further comprise a third longitudinal wire between the first and second longitudinal wires. In an example, the third longitudinal wire can be undulating or sinusoidal. In an example, the third longitudinal wire can be sinusoidal and out-of-phase with the first and second longitudinal wires. In an example, the device can be asymmetric with respect to its longitudinal axis. In an example, the device can be asymmetric with respect to its longitudinal axis and there is alternating side-to-side variation in this longitudinal asymmetry in different locations along its longitudinal axis.

I claim:

1. A device for occluding an aneurysm comprising:
a first longitudinal wire which is inserted into an aneurysm sac;
a second longitudinal wire which is inserted into the aneurysm sac, wherein the first and second longitudinal wires intersect, overlap, or connect at least three times along their longitudinal axes, forming at least two wire loops between the first and second longitudinal wires; and
a mesh material which spans the at least two loops.

2. The device in claim 1 wherein the first and second longitudinal wires are undulating.

3. The device in claim 1 wherein the first and second longitudinal wires are sinusoidal.

4. The device in claim 1 wherein the first and second longitudinal wires are sinusoidal and out-of-phase with each other.

5. The device in claim 1 wherein the device further comprises a third longitudinal wire between the first and second longitudinal wires.

6. The device in claim 5 wherein the third longitudinal wire is undulating.

7. The device in claim 5 wherein the third longitudinal wire is sinusoidal.

8. The device in claim 5 wherein the third longitudinal wire is sinusoidal and out-of-phase with the first and second longitudinal wires.

9. The device in claim 1 wherein the device is asymmetric with respect to its longitudinal axis.

10. The device in claim 9 wherein the device is asymmetric with respect to its longitudinal axis and there is alternating side-to-side variation in this longitudinal asymmetry in different locations along its longitudinal axis.

11. A device for occluding an aneurysm comprising:
a first longitudinal wire which is inserted into an aneurysm sac;
a second longitudinal wire which is inserted into the aneurysm sac, wherein the first and second longitudinal wires converge and diverge at least three times along their longitudinal axes, forming at least two arcuate areas between the first and second longitudinal wires; and
a mesh material which spans the at least two arcuate areas.

12. The device in claim 11 wherein the first and second longitudinal wires are undulating.

13. The device in claim 11 wherein the first and second longitudinal wires are sinusoidal.

14. The device in claim 11 wherein the first and second longitudinal wires are sinusoidal and out-of-phase with each other.

15. The device in claim 11 wherein the device further comprises a third longitudinal wire between the first and second longitudinal wires.

16. The device in claim 15 wherein the third longitudinal wire is undulating.

17. The device in claim 15 wherein the third longitudinal wire is sinusoidal.

18. The device in claim 15 wherein the third longitudinal wire is sinusoidal and out-of-phase with the first and second longitudinal wires.

19. The device in claim 11 wherein the device is asymmetric with respect to its longitudinal axis.

20. The device in claim 19 wherein the device is asymmetric with respect to its longitudinal axis and there is alternating side-to-side variation in this longitudinal asymmetry in different locations along its longitudinal axis.
